SEQUENCE LISTING (70 sequences)

SEQ ID NO: 1 - RNA, 26 nt, synthetic construct
  source 1..26: mol_type = other RNA
cagaaggccccaggccctgtcttacc

SEQ ID NO: 2 - RNA, 25 nt, synthetic construct
  source 1..25: mol_type = other RNA
ggccctgtcttaccagctgtctttg

SEQ ID NO: 3 - RNA, 25 nt, synthetic construct
  source 1..25: mol_type = other RNA
agctgtctttgggacgacatctgcc

SEQ ID NO: 4 - RNA, 25 nt, synthetic construct
  source 1..25: mol_type = other RNA
ttcagctgttgggggagaagaagga

SEQ ID NO: 5 - RNA, 25 nt, synthetic construct
  source 1..25: mol_type = other RNA
ctgtctttgggacgacatctgcctt

SEQ ID NO: 6 - RNA, 26 nt, synthetic construct
  source 1..26: mol_type = other RNA
gctgttgggggagaagaaggacaaaa

SEQ ID NO: 7 - RNA, 26 nt, synthetic construct
  source 1..26: mol_type = other RNA
cttacagaagaggaaactacattacc

SEQ ID NO: 8 - RNA, 25 nt, synthetic construct
  source 1..25: mol_type = other RNA
ctggcacatgaaggaagggatcacc

SEQ ID NO: 9 - RNA, 24 nt, synthetic construct
  source 1..24: mol_type = other RNA
ctgtggaaggtggagcctttgtag

SEQ ID NO: 10 - RNA, 24 nt, synthetic construct
  source 1..24: mol_type = other RNA
ccgaagcccttctcaccagtgcac

SEQ ID NO: 11 - RNA, 25 nt, synthetic construct
  source 1..25: mol_type = other RNA
agggctctgaagttctctgagggtc

SEQ ID NO: 12 - RNA, 26 nt, synthetic construct
  source 1..26: mol_type = other RNA
tctgagggtcgaagagaaacacaggt

SEQ ID NO: 13 - RNA, 25 nt, synthetic construct
  source 1..25: mol_type = other RNA
ccagtgcacagggctctgaagttct

SEQ ID NO: 14 - RNA, 24 nt, synthetic construct
  source 1..24: mol_type = other RNA
aagcccttctcaccagtgcacagg

SEQ ID NO: 15 - RNA, 24 nt, synthetic construct
  source 1..24: mol_type = other RNA
cccttctcaccagtgcacagggct

SEQ ID NO: 16 - RNA, 24 nt, synthetic construct
  source 1..24: mol_type = other RNA
tagccgaagcccttctcaccagtg

SEQ ID NO: 17 - RNA, 25 nt, synthetic construct
  source 1..25: mol_type = other RNA
tttgtagccgaagcccttctcacca

SEQ ID NO: 18 - RNA, 24 nt, synthetic construct
  source 1..24: mol_type = other RNA
cctttgtagccgaagcccttctca

SEQ ID NO: 19 - RNA, 26 nt, synthetic construct
  source 1..26: mol_type = other RNA
caccctagaggaggcccccactcacc

SEQ ID NO: 20 - RNA, 25 nt, synthetic construct
  source 1..25: mol_type = other RNA
ctggccccacgtgcttcagtgtaaa

SEQ ID NO: 21 - RNA, 25 nt, synthetic construct
  source 1..25: mol_type = other RNA
gttctcgtcaggaaagcggcttccg

SEQ ID NO: 22 - RNA, 25 nt, synthetic construct
  source 1..25: mol_type = other RNA
tagatggacttcccgcctgtgccat

SEQ ID NO: 23 - RNA, 25 nt, synthetic construct
  source 1..25: mol_type = other RNA
tgtggttggtgaagtcgcccgcctg

SEQ ID NO: 24 - RNA, 26 nt, synthetic construct
  source 1..26: mol_type = other RNA
cctgtgagcacaaagcaccttctgag

SEQ ID NO: 25 - RNA, 25 nt, synthetic construct
  source 1..25: mol_type = other RNA
cattgtggttggtgaagtcgcccgc

SEQ ID NO: 26 - RNA, 25 nt, synthetic construct
  source 1..25: mol_type = other RNA
ccgtagatggacttcccgcctgtgc

SEQ ID NO: 27 - RNA, 25 nt, synthetic construct
  source 1..25: mol_type = other RNA
cttccgtagatggacttcccgcctg

SEQ ID NO: 28 - RNA, 25 nt, synthetic construct
  source 1..25: mol_type = other RNA
cggcttccgtagatggacttcccgc

SEQ ID NO: 29 - RNA, 25 nt, synthetic construct
  source 1..25: mol_type = other RNA
atggacttcccgcctgtgccattgt

SEQ ID NO: 30 - RNA, 25 nt, synthetic construct
  source 1..25: mol_type = other RNA
gacttcccgcctgtgccattgtggt

SEQ ID NO: 31 - RNA, 25 nt, synthetic construct
  source 1..25: mol_type = other RNA
ttcccgcctgtgccattgtggttgg

SEQ ID NO: 32 - RNA, 26 nt, synthetic construct
  source 1..26: mol_type = other RNA
agagggcctggggcagggaactcacc

SEQ ID NO: 33 - RNA, 25 nt, synthetic construct
  source 1..25: mol_type = other RNA
cagtctgtctttatggtgcagatga

SEQ ID NO: 34 - RNA, 25 nt, synthetic construct
  source 1..25: mol_type = other RNA
agaactgggagccgttggtgttagg

SEQ ID NO: 35 - RNA, 26 nt, synthetic construct
  source 1..26: mol_type = other RNA
accagcattagccatggacaggacac

SEQ ID NO: 36 - RNA, 26 nt, synthetic construct
  source 1..26: mol_type = other RNA
cctgcaaaaaccagcagggctgagcc

SEQ ID NO: 37 - RNA, 25 nt, synthetic construct
  source 1..25: mol_type = other RNA
agatgaagaactgggagccgttggt

SEQ ID NO: 38 - RNA, 25 nt, synthetic construct
  source 1..25: mol_type = other RNA
actgggagccgttggtgttaggacc

SEQ ID NO: 39 - RNA, 25 nt, synthetic construct
  source 1..25: mol_type = other RNA
gggagccgttggtgttaggaccagc

SEQ ID NO: 40 - RNA, 25 nt, synthetic construct
  source 1..25: mol_type = other RNA
agccgttggtgttaggaccagcatt

SEQ ID NO: 41 - RNA, 25 nt, synthetic construct
  source 1..25: mol_type = other RNA
cgttggtgttaggaccagcattagc

SEQ ID NO: 42 - RNA, 26 nt, synthetic construct
  source 1..26: mol_type = other RNA
aggaccagcattagccatggacagga

SEQ ID NO: 43 - RNA, 26 nt, synthetic construct
  source 1..26: mol_type = other RNA
gttaggaccagcattagccatggaca

SEQ ID NO: 44 - RNA, 26 nt, synthetic construct
  source 1..26: mol_type = other RNA
ggtgttaggaccagcattagccatgg

SEQ ID NO: 45 - RNA, 25 nt, synthetic construct
  source 1..25: mol_type = other RNA
attccacgaccgcgttatgaccgag

SEQ ID NO: 46 - DNA, 19 bp, synthetic construct
  source 1..19: mol_type = other DNA
gacccgtcctcttcctcct

SEQ ID NO: 47 - DNA, 20 bp, synthetic construct
  source 1..20: mol_type = other DNA
agctcaactggccacagtct

SEQ ID NO: 48 - DNA, 19 bp, synthetic construct
  source 1..19: mol_type = other DNA
gcagatgtcgtcccaaaga

SEQ ID NO: 49 - DNA, 19 bp, synthetic construct
  source 1..19: mol_type = other DNA
ttgacgtgaccgaacacaa

SEQ ID NO: 50 - DNA, 19 bp, synthetic construct
  source 1..19: mol_type = other DNA
cgtgtacctggacgtggac

SEQ ID NO: 51 - DNA, 19 bp, synthetic construct
  source 1..19: mol_type = other DNA
gagccgttggtgttaggac

SEQ ID NO: 52 - DNA, 20 bp, synthetic construct
  source 1..20: mol_type = other DNA
ggcttccgtagatggacttc

SEQ ID NO: 53 - DNA, 50 bp, Homo sapiens
  source 1..50: mol_type = genomic DNA
gccgggaacctgggcaagccaataaaggctgcggcgcgcggctgcgcggg

SEQ ID NO: 54 - DNA, 275 bp, Homo sapiens
  source 1..275: mol_type = genomic DNA
actcggccttctgggcgcgcgcgacgtcagtttgagttctgtgttctccccgcccgtgtcccgcccgacccgcgcccgcgatgctggcgctgcgctgcggctcccgctggctcggcctgctctccgtcccgcgctccgtgccgctgcgcctccccgcggcccgcgcctgcagcaagggctccggcgacccgtcctcttcctcctcctccgggaacccgctcgtgtacctggacgtggacgccaacgggaagccgctcggccgcgtggtgctggag

SEQ ID NO: 55 - DNA, 81 bp, Homo sapiens
  source 1..81: mol_type = genomic DNA
ttttgtccttcttctcccccaacagctgaaggcagatgtcgtcccaaagacagctggtaagacagggcctggggccttctg

SEQ ID NO: 56 - DNA, 139 bp, Homo sapiens
  source 1..139: mol_type = genomic DNA
acctgtgtttctcttcgaccctcagagaacttcagagccctgtgcactggtgagaagggcttcggctacaaaggctccaccttccacagggtgatcccttccttcatgtgccaggtaatgtagtttcctcttctgtaag

SEQ ID NO: 57 - DNA, 147 bp, Homo sapiens
  source 1..147: mol_type = genomic DNA
ctcagaaggtgctttgtgctcacaggcgggcgacttcaccaaccacaatggcacaggcgggaagtccatctacggaagccgctttcctgacgagaactttacactgaagcacgtggggccaggtgagtgggggcctcctctagggtg

SEQ ID NO: 58 - DNA, 126 bp, Homo sapiens
  source 1..126: mol_type = genomic DNA
ggctcagccctgctggtttttgcaggtgtcctgtccatggctaatgctggtcctaacaccaacggctcccagttcttcatctgcaccataaagacagactggtgagttccctgccccaggccctct

SEQ ID NO: 59 - DNA, 1628 bp, Homo sapiens
  source 1..1628: mol_type = genomic DNA
gttggatggcaagcatgttgtgttcggtcacgtcaaagagggcatggacgtcgtgaagaaaatagaatctttcggctctaagagtgggaggacatccaagaagattgtcatcacagactgtggccagttgagctaatctgtggccagggtgctggcatggtggcagctgcaaatgtccatgcacccaggtggccgcgttgggctgtcagccaaggtgcctgaaacgatacgtgtgcccactccactgtcacagtgtgcctgaggaaggctgctagggatgttagacctcggccaggacccaccacattgcttcctaatacccacccttcctcacgacctcatttctgggcatctttgtggacatgatgtcacccaccccttgtcaagcattgcctgtgattgcccagcccagattcatctgtgccttggacatggtgatggtgatgggttgccatccaagtgaaagtcttttccttgaccaagggggacagtcagttttgcaaaaggactctaatacctgtttaatattgtcttcctaattgggataatttaattaacaagattgactagaagtgaaactgcaacactaacttccccgtgctgtggtgtgacctgagttggtgacacaggccacagaccccagagcttggcttttgaaacacaactcagggcttttgtgaaggttcccccgctgagatctttcctcctggttactgtgaagcctgttggtttgctgctgtcgtttttgaggagggcccatgggggtaggagcagttgaacctgggaacaaacctcacttgagctgtgcctagacaatgtgaattcctgtgttgctaacagaagtggcctgtaagctcctgtgctccggagggaagcatttcctggtaggctttgatttttctgtgtgttaaagaaattcaatctactcatgatgtgttatgcataaaacatttctggaacatggatttgtgttcaccttaaatgtgaaaataaatcctattttctatggaagactggtacctggtttctggaagaggggtctgtgacttggagctgatctttactgagctcgccgtggcagatgccatgctcaggacgttcatgtggatggtttcatgtcatcgtgctggcaacttgtcctccctgccttagagatgaggctcagacaaacgaccttagcacccatagcctatgccatgagcactggctccaccctgaatcccagctcctccccttagtgaccccaagtctgtttccctcagctgcataaggaggcgatatagtttgaatatttgtccccagccaaatctcatgttgaactgtaatccccagtgctggaggtggggcctgctacgaggtgtttggatcatggggacgggtatttcatggcttggtgctgttttcttgatggtgaattattgcaagatacggtcatttaaaattgtgtggcacctccccctgcccccttcttgctcctgctttcaccatgtgacatgcctgatcccccttcaccttttgccatggtcataagcttcctgaggcctccctggaagctgagcagatgccagcaccatgcttcctgtacatcctgcagaaccataagccaattaaacctttttaataataaa

SEQ ID NO: 60 - DNA, 50 bp, Homo sapiens
  source 1..50: mol_type = genomic DNA
ttactcagtctcaagtatttttttatagcaatacaagaatcctaacacgg

SEQ ID NO: 61 - DNA, 25 bp, Homo sapiens
  source 1..25: mol_type = genomic DNA
gtgagaccgctcgcagggccggcct

SEQ ID NO: 62 - DNA, 25 bp, Homo sapiens
  source 1..25: mol_type = genomic DNA
ttttgtccttcttctcccccaacag

SEQ ID NO: 63 - DNA, 25 bp, Homo sapiens
  source 1..25: mol_type = genomic DNA
gtaagacagggcctggggccttctg

SEQ ID NO: 64 - DNA, 25 bp, Homo sapiens
  source 1..25: mol_type = genomic DNA
acctgtgtttctcttcgaccctcag

SEQ ID NO: 65 - DNA, 25 bp, Homo sapiens
  source 1..25: mol_type = genomic DNA
gtaatgtagtttcctcttctgtaag

SEQ ID NO: 66 - DNA, 25 bp, Homo sapiens
  source 1..25: mol_type = genomic DNA
ctcagaaggtgctttgtgctcacag

SEQ ID NO: 67 - DNA, 25 bp, Homo sapiens
  source 1..25: mol_type = genomic DNA
gtgagtgggggcctcctctagggtg

SEQ ID NO: 68 - DNA, 25 bp, Homo sapiens
  source 1..25: mol_type = genomic DNA
ggctcagccctgctggtttttgcag

SEQ ID NO: 69 - DNA, 25 bp, Homo sapiens
  source 1..25: mol_type = genomic DNA
gtgagttccctgccccaggccctct

SEQ ID NO: 70 - DNA, 25 bp, Homo sapiens
  source 1..25: mol_type = genomic DNA
tcacccgggtcacccgtcctcacag

(19) United States
(12) Patent Application Publication
(10) Pub. No.: US 2025/0340873 A1
(43) Pub. Date: Nov. 6, 2025

(54) Method for Treating Cyclophilin D Associated Diseases

(71) Applicant: Resonance Health Analysis Services Pty Ltd, Burswood (AU)

(72) Inventor: Sherif Boulos, Burswood (AU)

(21) Appl. No.: 18/710,452

(22) PCT Filed: Nov. 17, 2022
(86) PCT No.: PCT/AU2022/051375

§ 371 Date: May 15, 2024

(30) Foreign Application Priority Data
Nov. 18, 2021 (AU) 2021903724

(51) Int. Cl. C12N 15/113

(52) U.S. Cl. CPC C12N 15/113; C12N 2310/14; C12N 2310/321; C12N 2320/33

(57) ABSTRACT

An isolated or purified antisense oligomer which has a modified backbone structure for modifying pre-mRNA splicing in the PPIF gene transcript or part thereof.

### INCORPORATION-BY-REFERENCE OF MATERIAL SUBMITTED IN ELECTRONIC FORM

Applicant hereby incorporates by reference the Sequence Listing material filed in electronic form herewith. This file is labelled “WRA122seq.xml” (65, 141 bytes in size and created Nov. 27, 2024).

### TECHNICAL FIELD

The present invention relates to the use of antisense oligomers to treat, prevent or ameliorate the effects of a diseases and pathologies associated with Cyclophilin D.

### BACKGROUND ART

Cyclophilin D (CYPD, PPIF) is a ubiquitously distributed protein belonging to the immunophilin family, whose members catalyse the cis-trans isomerization of proline imidic peptide bonds. In humans, the PPIF gene is located on chromosome 10 wherein it encodes a 17.5-kDa protein of 207 amino acids comprising six exons. Associated with the mitochondria, CYPD is implicated in the opening of the mitochondrial permeability transition pore (mPTP) (PubMed: 26387735). Under stress, such as increased mitochondrial Ca²⁺ influx, CYPD is thought to bind to the adenine nucleotide translocase (ANT) machinery to facilitate opening of the mPTP pore. In this manner, CYPD is regarded as an important mediator in the release of pro-apoptotic and other factors from the mitochondria that leads to cell death.

Structurally, CYPD shows a high degree of homology with other members of the cyclophilin family in its core β-barrel/isomerase region, which contains a surface hydrophobic pocket that constitutes the proline binding motif. Both N- and C-termini of CYPD differ significantly from other cyclophilin family members.

CYPD has been shown to play a critical function in a range of human diseases including ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease.

Currently, CYPD is modulated in a variety of diseases and pathologies by administration of naturally occurring immunosuppressive drugs such as cyclosporin A (CsA) and their derivatives known to be active against CYPD. However, the use of pan-cyclophilin inhibitors such as CsA or its synthetic derivatives, may not be ideal, as their use can lead to the inactivation of cyclophilin members whose action(s) are beneficial and unrelated to the disease being treatment.

There is a need to provide new treatments or preventative measures for modulating the levels of CYPD in both specific tissues and the body as a whole; or at least the provision of methods to compliment the previously known treatments. The present invention seeks to provide an improved or alternative method for treating, preventing or ameliorating the effects of diseases and pathologies associated with cyclophilin D.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

Broadly, according to one aspect of the invention, there is provided an isolated or purified antisense oligomer for modifying pre-mRNA splicing in the PPIF gene transcript or part thereof. Preferably, there is provided an isolated or purified antisense oligomer for inducing non-productive splicing in the PPIF gene transcript or part thereof.

For example, in one aspect of the invention, there is provided an antisense oligomer of 10 to 50 nucleotides comprising a targeting sequence complementary to a region near or within an intron of the PPIF gene transcript or part thereof. In another aspect of the invention, there is provided an antisense oligomer of 10 to 50 nucleotides comprising a targeting sequence complementary to a region near or within an exon of the PPIF gene transcript or part thereof.

Preferably, the antisense oligomer is selected from the group comprising the sequences set forth in Table 3. Preferably, the antisense oligomer is selected from the list comprising: SEQ ID NOs: 1-44, more preferably SEQ ID NOs: 35 or 37.

The antisense oligomer preferably operates to induce skipping of one or more of the exons of the PPIF gene transcript or part thereof. For example, the antisense oligomer may induce skipping of exons 3, and/or 4.

The antisense oligomer of the invention may be selected to be an antisense oligomer capable of binding to a selected PPIF target site, wherein the target site is an mRNA splicing site selected from a splice donor site, splice acceptor sites, or exonic splicing elements. The target site may also include some flanking intronic sequences when the donor or acceptor splice sites are targeted.

More specifically, the antisense oligomer may be selected from the group comprising of any one or more of SEQ ID NOs: 1-44, more preferably SEQ ID NOs: 35 or 37; and/or the sequences set forth in Table 3, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a PPIF gene transcript In certain embodiments, antisense oligomers may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

The invention extends also to a combination of two or more antisense oligomers capable of binding to a selected target to induce exon exclusion in a PPIF gene transcript, including a construct comprising two or more such antisense oligomers. The construct may be used for an antisense oligomer-based therapy.

The invention extends, according to a still further aspect thereof, to cDNA or cloned copies of the antisense oligomer sequences of the invention, as well as to vectors containing the antisense oligomer sequences of the invention. The invention extends further also to cells containing such sequences and/or vectors.

There is also provided a method for modulating splicing in a PPIF gene transcript, the method including the step of:
a) providing one or more of the antisense oligomers as described herein and allowing the oligomer(s) to bind to a target nucleic acid site.

There is also provided a pharmaceutical, prophylactic, or therapeutic composition to treat, prevent or ameliorate the effects of a disease or pathology related to PPIF gene expression in a patient, the composition comprising:
a) one or more antisense oligomers as described herein; and
b) one or more pharmaceutically acceptable carriers and/or diluents.

The composition may comprise about 1 nM to 1000 nM of each of the desired antisense oligomer(s) of the invention. Preferably, the composition may comprise about 10 nM to 500 nM, most preferably between 1 nM and 10 nM of each of the antisense oligomer(s) of the invention.

There is also provided a method to treat, prevent or ameliorate the effects of a disease or pathology associated with PPIF gene expression, comprising the step of:
a) administering to the patient an effective amount of one or more antisense oligomers or pharmaceutical composition comprising one or more antisense oligomers as described herein.

There is also provided the use of purified and isolated antisense oligomers as described herein, for the manufacture of a medicament to treat, prevent or ameliorate the effects of a disease or pathology associated with PPIF gene expression.

There is also provided a kit to treat, prevent or ameliorate the effects of a disease or pathology associated with PPIF gene expression in a patient, which kit comprises at least an antisense oligomer as described herein and combinations or cocktails thereof, packaged in a suitable container, together with instructions for its use.

Preferably the disease or pathology associated with PPIF gene expression in a patient is chosen from the list comprising: ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease.

The subject with the disease or pathology associated with PPIF gene expression may be a mammal, including a human.

Further aspects of the invention will now be described with reference to the accompanying non-limiting examples and drawings.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1. RT-PCR analysis of total RNA harvested from HepG2 cultures at 24 hours post-transfection with ASOs (at 500 nM and synthesised with a 2-O-Methyl backbone) designed to induce skipping of exon 2 or 3 from the full-length CYPD mRNA sequence. Agarose Gel Electrophoresis (AGE) of the resulting PCR products showed that ASOs CDm2.1, CDm2.2, CDm2.3 and CDm2.5 (FIGS. 1a and 1b), induced the deletion of a 31 bp fragment, consistent with the size of exon 2, while ASOs CDm3.3 and CDm3.4 induced the deletion of a 89 bp fragment, consistent with the size of exon 3 (FIGS. 1b and 1c). A 100 bp molecular weight marker was loaded in the first lane.

FIG. 2. RT-PCR analysis of total RNA harvested from HepG2 cultures at 24 hours post-transfection with ASOs (at 500 nM and synthesised with a 2-O-Methyl backbone) designed to induce skipping of exon 4 or 5 from the full-length CYPD mRNA sequence. Agarose Gel Electrophoresis (AGE) of the resulting PCR products showed that ASOs CDm4.3, CDm4.4, CDm4.5, and CDm4.7 (FIGS. 2a and 2b) significantly induced the deletion of a 97 bp fragment, consistent with the size of exon 4, while ASOs CDm5.3 and CDm 5.4 induced the deletion of a 76 bp fragment, consistent with the size of exon 5 (FIGS. 2b and 2c). A 100 bp molecular weight marker was loaded in the first and last lanes.

FIG. 3. RT-PCR analysis of total RNA harvested from HepG2 cultures at 24 hours post-transfection with ASOs (at 250 nM, synthesised with a MOE backbone and referred to as CDm3.41, CDm3.42, CDm3.43, CDm3.44 and CDm3.45) derived by ‘microwalking’ 5′ and 3′ of the CDm3.4 sequence. Agarose Gel Electrophoresis (AGE) of PCR products (derived using primers SEQ ID 49/50) showed that each ASO induced variable exon 3 deletion, with ASO CDm3.45 inducing near complete skipping. NTC denotes a mock transfection control. A 100 bp molecular weight marker was loaded in the first lane.

FIG. 4. RT-PCR analysis of total RNA harvested from HepG2 cultures at 24 hours post-transfection with ASOs (at 250 nM, synthesised with a MOE backbone and referred to as CDm4.41, CDm4.42, CDm4.43, CDm4.44 CDm4.45 and CDm4.46) derived by ‘microwalking’ 5′ and 3′ of the CDm4.4 sequence. Agarose Gel Electrophoresis (AGE) of PCR products (derived using primers SEQ ID 49/50) showed that all ASOs induced substantial exon 4 deletion, with ASO CDm 4.4 and CDm4.41 inducing near complete skipping as evidenced by the lack of any residual FL CYPD mRNA. NTC denotes a mock transfection control. A 100 bp molecular weight marker was loaded in the first lane.

FIG. 5. RT-PCR analysis of total RNA harvested from HepG2 cultures at 24 hours post-transfection with ASOs (at 250 nM, synthesised with a MOE backbone and referred to as CDm5.31, 5.32, 5.33, 5.34 and 5.32; and CDm5.41, 5.42 and 5.43) derived by ‘microwalking’ 5′ and 3′ of the CDm5.3 and 5.4 sequences. Agarose Gel Electrophoresis (AGE) of PCR products (derived using primers SEQ ID 49/50) showed that all ASOs induced almost complete exon 5 deletion. NTC denotes a mock transfection control. A 100 bp molecular weight marker was loaded in the first lane.

FIG. 6. Effect of ASO treatment on CYPD mRNA and Protein expression. A) RT-PCR of total RNA harvested from HepG2 cultures at 24 hours post-transfection with ASOs (at 250 nM synthesised with a MOE backbone) CDm3.45, CDm4.4, CDm4.41, CDm 5.4, CDm5.41, CDm5.42, CDm5.3, CDm5.31 and the scrambled control ASO 953 and showing deletion of either exon 3, 4 or 5 from full-length CYPD mRNA: a) Agarose Gel Electrophoresis (AGE) of PCR products (derived using primers SEQ ID 48 and 49) showed the deletion of a 94 bp fragment, consistent with the expected size of exon 3, while the ASO scrambled control 953 did not induce exon 3 skipping; b) Image of Western blots of lysates harvested 3 days post transfection and probed with anti-CYPD antibody; c) Plot showing percent CYPD band intensity (relative to total protein loading).

### DESCRIPTION OF INVENTION

### Detailed Description of the Invention

### Antisense Oligonucleotides

Cyclophilin D (CYPD), also known as peptidylprolyl isomerase F (PPIF), is an enzymatic protein that in humans is encoded by the PPIF gene on chromosome 10. In the present application, the terms CYPD and PPIF are used interchangeably to represent the cyclophilin B gene and protein. The cyclophilin D referred to herein is not to be confused with Cyclophilin-40 which has also been referred to as CYPD.

Expression of the CYPD protein is associated with a range of inflammatory diseases and cancers. CYPD plays a vital role in many disease-related processes that give rise to cardiovascular disease (cardiac arrest, ischaemia reperfusion injury), cerebrovascular disease (stroke, traumatic brain injury, spinal contusion injury and ischaemia reperfusion injury), liver diseases, kidney diseases, diabetes, neurodegeneration (Alzheimer's disease, Parkinson's disease, ALS, traumatic brain injury, spinal injury and multiple sclerosis), cancer and aging.

Given its mitochondrial role in regulating cellular redox responses to external stimuli, CYPD is pro-inflammatory. As CYPD has been detected in serum it could interact with the CD147 (the chief cell receptor for CYPD) receptor (in a manner similar to cyclophilin A and cyclophilin B) to induce chemotactic activity in macrophages, neutrophils, and leukocytes.

Diseases and pathologies associated with expression of the CYPD protein can be found in the table below, along with postulated mechanisms of actions revealed in the respective studies undertaken.

TABLE 1
Diseases and pathologies associated with expression of the CYPD protein

| Associated | | Disease related | |
|---|---|---|---|
| Disease(s) | Organ | pathway | PMID |
| Mycardial | | Induction of the mitochondrial MTP pore | 33717647 |
| infarction | | opening to initiate programmed cell death | |
| Ischaemia | liver, heart, | Induction of the mitochondrial MTP pore | 33621193, |
| repurfusion | brain, kidney, | opening to initiate programmed cell death. | 32879367, |
| injury | lung | | 32925170, |
| | | | 33050736 |
| Non- | liver | Induction of the mitochondrial MTP pore opening to initiate | 34434058 |
| alcoholic | | programmed cell death. Elevated serum levels could be | |
| fatty liver | | involved in immune activation via CD147 or other receptors. | |
| disease | | Associated with higher fibrosis scores in type 2 diabetes | |
| Stroke | brain | Induction of the mitochondrial MTP pore | 32605939 |
| | | opening to initiate programmed cell death. | |
| | | Excitotoxic related neuronal cell death | |
| Traumatic | brain | Induction of the mitochondrial MTP pore | 33498273 |
| Brain Injury | | opening to initiate programmed cell death. | |
| Alzheimer's | brain | Induction of the mitochondrial MTP pore | 33679286, |
| disease and | | opening to initiate programmed cell death. | 32825572, |
| cognitive | | Tau associated cell damage is influenced | |
| decline | | by CYPD | |
| Parkinson's | | Induction of the mitochondrial MTP pore | 32576835 |
| disease | | opening to initiate programmed cell death. | |
| | | CYPD interacts with alpha-synuclein and | |
| | | affects its aggregation. | |
| Motor Neuron | | Induction of the mitochondrial MTP pore | |
| Disease | | opening to initiate programmed cell death | |
| Mitochondrial | multiple | Induction of the mitochondrial MTP pore | 33000352 |
| Diseases | organs | opening to initiate programmed cell death | |
| mitochondrial | skeletal | Induction of the mitochondrial MTP pore | |
| myopathy | muscle | opening to initiate programmed cell death | |
| Metabolic | systemic, | Induction of the mitochondrial MTP pore | 34455488 |
| Disease and | skeletal and | opening to initiate programmed cell death; | |
| Diabetes | pancreas | Elevated serum levels could be involved | |
| | | in immune activation via CD147 or other receptors | |
| Pancreatitis | | Induction of the mitochondrial MTP pore | 31631343 |
| | | opening to initiate programmed cell death | |
| Lymphoblastic | | | 34445394 |
| leukemia | | | |
| Kidney | kidney | Induction of the mitochondrial MTP pore opening | 34396791 |
| Disease | | to initiate programmed cell death. Fibrogenesis | |
| Aging | brain | Induction of the mitochondrial MTP pore opening | 33574977 |
| | | to initiate programmed cell death. Neuronal damage | |
| Muscle | skeletal | In Duchenne Muscular Dystrophy mitochondrial | 33228255 |
| disease | muscle | related changes occur in skeletal muscle. | |
| Transplant | liver, heart, | Induction of the mitochondrial MTP pore | 33374660, |
| rejection | kidney | opening to initiate programmed cell death. | 30203531 |
| | | Inhibition of CYPD can inhibit | |
| | | necroptotic cell death | |
| Thalassemia | heart | Iron overload related cell death | 32824344 |

TABLE 2
Human Cyclophilin D Exon Table

| SEQ | | | | | | |
|---|---|---|---|---|---|---|
| ID NO: | No. | Exon/Intron | Start | End | Length | Sequence |
| 53 | | 5′ upstream sequence | | | | gccgggaacctgggcaagccaataaaggctg |
| | | | | | | cggcgcgcggctgcgcggg |
| 54 | 1 | ENSE00003503 | 79,347,469 | 79,347,743 | 275 | ACTCGGCCTTCTGGGCGCGCGCGACGTCAGT |
| | | 708 | | | | TTGAGTTCTGTGTTCTCCCCGCCCGTGTCCC |
| | | | | | | GCCCGACCCGCGCCCGCGATGCTGGCGCTGC |
| | | | | | | GCTGCGGCTCCCGCTGGCTCGGCCTGCTCTC |
| | | | | | | CGTCCCGCGCTCCGTGCCGCTGCGCCTCCCC |
| | | | | | | GCGGCCCGCGCCTGCAGCAAGGGCTCCGGCG |
| | | | | | | ACCCGTCCTCTTCCTCCTCCTCCGGGAACCC |
| | | | | | | GCTCGTGTACCTGGACGTGGACGCCAACGGG |
| | | | | | | AAGCCGCTCGGCCGCGTGGTGCTGGAG |
| 55 | 2 | ENSE00003637 | 79,349,076 | 79,349,106 | 31 | ttttgtccttcttctcccccaacagCTGAAG |
| | | 288 | | | | GCAGATGTCGTCCCAAAGACAGCTGgtaaga |
| | | | | | | cagggcctggggccttctg |
| 56 | 3 | ENSE00003506 | 79,349,665 | 79,349,753 | 89 | acctgtgtttctcttcgaccctcagAGAACT |
| | | 032 | | | | TCAGAGCCCTGTGCACTGGTGAGAAGGGCTT |
| | | | | | | CGGCTACAAAGGCTCCACCTTCCACAGGGTG |
| | | | | | | ATCCCTTCCTTCATGTGCCAGgtaatgtagt |
| | | | | | | ttcctcttctgtaag |
| 57 | 4 | ENSE00000429 | 79,351,487 | 79,351,583 | 97 | ctcagaaggtgctttgtgctcacagGCGGGC |
| | | 803 | | | | GACTTCACCAACCACAATGGCACAGGCGGGA |
| | | | | | | AGTCCATCTACGGAAGCCGCTTTCCTGACGA |
| | | | | | | GAACTTTACACTGAAGCACGTGGGGCCAGgt |
| | | | | | | gagtgggggcctcctctagggtg |
| 58 | 5 | ENSE00003587 | 79,352,317 | 79,352,392 | 76 | ggctcagccctgctggtttttgcagGTGTCC |
| | | 434 | | | | TGTCCATGGCTAATGCTGGTCCTAACACCAA |
| | | | | | | CGGCTCCCAGTTCTTCATCTGCACCATAAAG |
| | | | | | | ACAGACTGgtgagttccctgccccaggccct |
| | | | | | | ct |
| 59 | 6 | ENSE00001136 | 79,353,707 | 79,355,334 | 1,628 | GTTGGATGGCAAGCATGTTGTGTTCGGTCAC |
| | | 191 | | | | GTCAAAGAGGGCATGGACGTCGTGAAGAAAA |
| | | | | | | TAGAATCTTTCGGCTCTAAGAGTGGGAGGAC |
| | | | | | | ATCCAAGAAGATTGTCATCACAGACTGTGGC |
| | | | | | | CAGTTGAGCTAATCTGTGGCCAGGGTGCTGG |
| | | | | | | CATGGTGGCAGCTGCAAATGTCCATGCACCC |
| | | | | | | AGGTGGCCGCGTTGGGCTGTCAGCCAAGGTG |
| | | | | | | CCTGAAACGATACGTGTGCCCACTCCACTGT |
| | | | | | | CACAGTGTGCCTGAGGAAGGCTGCTAGGGAT |
| | | | | | | GTTAGACCTCGGCCAGGACCCACCACATTGC |
| | | | | | | TTCCTAATACCCACCCTTCCTCACGACCTCA |
| | | | | | | TTTCTGGGCATCTTTGTGGACATGATGTCAC |
| | | | | | | CCACCCCTTGTCAAGCATTGCCTGTGATTGC |
| | | | | | | CCAGCCCAGATTCATCTGTGCCTTGGACATG |
| | | | | | | GTGATGGTGATGGGTTGCCATCCAAGTGAAA |
| | | | | | | GTCTTTTCCTTGACCAAGGGGGACAGTCAGT |
| | | | | | | TTTGCAAAAGGACTCTAATACCTGTTTAATA |
| | | | | | | TTGTCTTCCTAATTGGGATAATTTAATTAAC |
| | | | | | | AAGATTGACTAGAAGTGAAACTGCAACACTA |
| | | | | | | ACTTCCCCGTGCTGTGGTGTGACCTGAGTTG |
| | | | | | | GTGACACAGGCCACAGACCCCAGAGCTTGGC |
| | | | | | | TTTTGAAACACAACTCAGGGCTTTTGTGAAG |
| | | | | | | GTTCCCCCGCTGAGATCTTTCCTCCTGGTTA |
| | | | | | | CTGTGAAGCCTGTTGGTTTGCTGCTGTCGTT |
| | | | | | | TTTGAGGAGGGCCCATGGGGGTAGGAGCAGT |
| | | | | | | TGAACCTGGGAACAAACCTCACTTGAGCTGT |
| | | | | | | GCCTAGACAATGTGAATTCCTGTGTTGCTAA |
| | | | | | | CAGAAGTGGCCTGTAAGCTCCTGTGCTCCGG |
| | | | | | | AGGGAAGCATTTCCTGGTAGGCTTTGATTTT |
| | | | | | | TCTGTGTGTTAAAGAAATTCAATCTACTCAT |
| | | | | | | GATGTGTTATGCATAAAACATTTCTGGAACA |
| | | | | | | TGGATTTGTGTTCACCTTAAATGTGAAAATA |
| | | | | | | AATCCTATTTTCTATGGAAGACTGGTACCTG |
| | | | | | | GTTTCTGGAAGAGGGGTCTGTGACTTGGAGC |
| | | | | | | TGATCTTTACTGAGCTCGCCGTGGCAGATGC |
| | | | | | | CATGCTCAGGACGTTCATGTGGATGGTTTCA |
| | | | | | | TGTCATCGTGCTGGCAACTTGTCCTCCCTGC |
| | | | | | | CTTAGAGATGAGGCTCAGACAAACGACCTTA |
| | | | | | | GCACCCATAGCCTATGCCATGAGCACTGGCT |
| | | | | | | CCACCCTGAATCCCAGCTCCTCCCCTTAGTG |
| | | | | | | ACCCCAAGTCTGTTTCCCTCAGCTGCATAAG |
| | | | | | | GAGGCGATATAGTTTGAATATTTGTCCCCAG |
| | | | | | | CCAAATCTCATGTTGAACTGTAATCCCCAGT |
| | | | | | | GCTGGAGGTGGGGCCTGCTACGAGGTGTTTG |
| | | | | | | GATCATGGGGACGGGTATTTCATGGCTTGGT |
| | | | | | | GCTGTTTTCTTGATGGTGAATTATTGCAAGA |
| | | | | | | TACGGTCATTTAAAATTGTGTGGCACCTCCC |
| | | | | | | CCTGCCCCCTTCTTGCTCCTGCTTTCACCAT |
| | | | | | | GTGACATGCCTGATCCCCCTTCACCTTTTGC |
| | | | | | | CATGGTCATAAGCTTCCTGAGGCCTCCCTGG |
| | | | | | | AAGCTGAGCAGATGCCAGCACCATGCTTCCT |
| | | | | | | GTACATCCTGCAGAACCATAAGCCAATTAAA |
| | | | | | | CCTTTTTAATAATAAA |
| 60 | | 3′ downstream sequence | | | | ttactcagtctcaagtatttttttatagcaa |
| | | | | | | tacaagaatcctaacacgg...... |
| 61 | | Intron 1-2 start | 79,347,744 | 79,349,075 | 1,332 | gtgagaccgctcgcagggccggcct...... |
| | | | | | | .... |
| 62 | | Intron 1-2 end | 79,347,744 | 79,349,075 | 1,332 | ttttgtccttcttctcccccaacag |
| 63 | | Intron 2-3 start | 79,349,107 | 79,349,664 | 558 | gtaagacagggcctggggccttctg...... |
| | | | | | | .... |
| 64 | | Intron 2-3 end | 79,349,107 | 79,349,664 | 558 | acctgtgtttctcttcgaccctcag |
| 65 | | Intron 3-4 start | 79,349,754 | 79,351,486 | 1,733 | gtaatgtagtttcctcttctgtaag...... |
| | | | | | | .... |
| 66 | | Intron 3-4 end | 79,349,754 | 79,351,486 | 1,733 | ctcagaaggtgctttgtgctcacag |
| 67 | | Intron 4-5 start | 79,351,584 | 79,352,316 | 733 | gtgagtgggggcctcctctagggtg...... |
| | | | | | | .... |
| 68 | | Intron 4-5 end | 79,351,584 | 79,352,316 | 733 | ggctcagccctgctggtttttgcag |
| 69 | | Intron 5-6 start | 79,352,393 | 79,353,706 | 1,314 | gtgagttccctgccccaggccctct...... |
| | | | | | | .... |

According to a first aspect of the invention, there is provided antisense oligomers capable of binding to a selected target on a PPIF gene transcript to modify pre-mRNA splicing in a PPIF gene transcript or part thereof. Broadly, there is provided an isolated or purified antisense oligomer for inducing targeted exon exclusion and/or terminal intron retention in a PPIF gene transcript or part thereof. Preferably, there is provided an isolated or purified antisense oligomer for inducing non-productive splicing in the PPIF gene transcript or part thereof.

In the present invention, antisense oligomers are also known as antisense oligonucleotides, AOSs, AONs and AONs—the terms are interchangeable.

In one aspect there is provided an antisense oligomer of 10 to 50 nucleotides comprising a targeting sequence complementary to a region near or within an intron of the PPIF gene transcript or part thereof. In another aspect of the invention, there is provided an antisense oligomer of 10 to 50 nucleotides comprising a targeting sequence complementary to a region near or within an exon of the PPIF gene transcript or part thereof.

In contrast to other antisense oligomer-based therapies, the present invention does not induce increased degradation of RNA via recruitment of RNase H, wherein the RNase H preferentially binds and degrades RNA bound in duplex to the DNA of the PPIF gene. Nor does it rely on hybridization of the antisense oligomer to the PPIF genomic DNA or the binding of antisense oligomers to mRNA to modulate the amount of CYPD protein produced by interfering with normal functions such as replication, transcription, translocation and translation.

Rather, the antisense oligomers are used to modify pre-mRNA splicing in a PPIF gene transcript or part thereof and induce exon “skipping” and/or terminal intron retention. The strategy preferably reduces total protein expression or generates proteins which lack functional domains, leading to reduced protein function.

By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

An antisense oligomer can be said to be “directed to” or “targeted against” a target sequence with which it hybridizes. In certain embodiments, the target sequence includes a region including a 3′ or 5′ splice site of a pre-processed mRNA, a branch point, or other sequences involved in the regulation of splicing. The target sequence may be within an exon or within an intron or spanning an intron/exon junction.

In certain embodiments, the antisense oligomer has sufficient sequence complementarity to a target RNA (i.e., the RNA for which splice site selection is modulated) to block a region of a target RNA (e.g., pre-mRNA) in an effective manner. In exemplary embodiments, such blocking of PPIF pre-mRNA serves to modulate splicing, either by masking a binding site for a native protein that would otherwise modulate splicing and/or by altering the structure of the targeted RNA. In some embodiments, the target RNA is target pre-mRNA (e.g., PPIF gene pre-mRNA).

An antisense oligomer having a sufficient sequence complementarity to a target RNA sequence to modulate splicing of the target RNA means that the antisense oligomer has a sequence sufficient to trigger the masking of a binding site for a native protein that would otherwise modulate splicing and/or alters the three-dimensional structure of the targeted RNA.

Selected antisense oligomers can be made shorter, e.g., about 12 bases, or longer, e.g., about 50 bases, and include a small number of mismatches, as long as the sequence is sufficiently complementary to effect splice modulation upon hybridization to the target sequence, and optionally forms with the RNA a heteroduplex having a Tm of 45° C. or greater.

Preferably, the antisense oligomer is selected from the group comprising the sequences set forth in Table 3. Preferably, the antisense oligomer is selected from the group comprising the sequences in SEQ ID NOs: 1-44, more preferably SEQ ID NOs: 35 or 37.

In certain embodiments, the degree of complementarity between the target sequence and antisense oligomer is sufficient to form a stable duplex. The region of complementarity of the antisense oligomers with the target RNA sequence may be as short as 8-11 bases, but can be 12-15 bases or more, e.g., 10-50 bases, 10-40 bases, 12-30 bases, 12-25 bases, 15-25 bases, 12-20 bases, or 15-20 bases, including all integers in between these ranges. An antisense oligomer of about 16-17 bases is generally long enough to have a unique complementary sequence. In certain embodiments, a minimum length of complementary bases may be required to achieve the requisite binding Tm, as discussed herein.

In certain embodiments, oligonucleotides as long as 50 bases may be suitable, where at least a minimum number of bases, e.g., 10-12 bases, are complementary to the target sequence. In general, however, facilitated or active uptake in cells is optimized at oligonucleotide lengths of less than about 30 bases. For example, for phosphorodiamidate morpholino oligomer (PMO) antisense oligomers, an optimum balance of binding stability and uptake generally occurs at lengths of 18-25 bases. Included are antisense oligomers (e.g., CPP-PMOs, PPMOs, PMOs, PMO-X, PNAs, LNAs, 2′-OMe, 2′MOE, 2′F oligomer, thiomorpholino and other hybrid oligomer chemistries) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases. (PMO-phosphorodiamidate morpholino oligomer; CPP-cell penetrating peptide; PPMO-peptide-conjugated phosphorodiamidate morpholino oligomer; PNA-peptide nucleic acid; LNA-locked nucleic acid; 2′-OMe-2′O-methyl-modified oligomer; 2′MOE-2′-O-methoxy ethyl oligomer, 2′F-2′ Fluoro)

In certain embodiments, antisense oligomers may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an antisense oligomer is not necessarily 100% complementary to the target sequence, it is effective to stably and specifically bind to the target sequence, such that splicing of the target pre-RNA is modulated.

The stability of the duplex formed between an antisense oligomer and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, antisense oligomers may have a binding Tm, with respect to a complementary-sequence RNA, of greater than body temperature and preferably greater than about 45° C. or 50° C. Tm's in the range 60-80° C. or greater are also included.

Additional examples of variants include antisense oligomers having about or at least about 70% sequence identity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, over the entire length of any of SEQ ID NOs: 1-44, more preferably SEQ ID NOs: 35 or 37, or the sequences provided in Table 3.

More specifically, there is provided an antisense oligomer capable of binding to a selected target site to modify pre-mRNA splicing in a PPIF gene transcript or part thereof. The antisense oligomer is preferably selected from those provided in Table X or SEQ ID NOs: 1-44, more preferably SEQ ID NOs: 35 or 37.

The modification of pre-mRNA splicing preferably induces “skipping”, or the removal of one or more exons or introns of the mRNA and/or terminal intron retention. The resultant protein may be of a shorter length when compared to the parent full-length CYPD protein due to either internal truncation or premature termination or may be longer due to terminal intron retention. These CYPD proteins may be termed isoforms of the unmodified CYPD protein.

The remaining exons of the mRNA generated may be in-frame and produce a shorter protein with a sequence that is similar to that of the parent full length protein, except that it has an internal truncation in a region between the original 3′ and 5′ ends. In another possibility, the exon skipping may induce a frame shift that results in a protein wherein the first part of the protein is substantially identical to the parent full length protein, but wherein the second part of the protein has a different sequence (eg a nonsense sequence) due to a frame-shift. Alternatively, the exon skipping may induce the production of a prematurely terminated protein due to a disruption of the reading frame and presence of a premature termination of translation. Additionally, the antisense oligomer may produce an artificially lengthened protein, due to in-frame terminal intron retention.

As exons 2, 3, 4 and 5 encode critical amino acids involved in the catalytic activity of CYPD, their deletion would be expected to lead to a non-functioning enzyme. Moreover, excision of exon 2 or 3 would create 6 or 4 premature stop codon(s) respectively, while excision of exon 4 or 5 would create 4 or 3 premature stop codon(s) respectively. Introducing aberrant stop codons in the mRNA would result in a non-functional, unstable and truncated polypeptide translated product. This truncated polypeptide would undergo rapid degradation, as evidenced by western blot analysis of ASO treated HepG2 cultures at three days post-transfection.

The removal of one or more exons may further lead to misfolding of the CYPD protein and a reduction in the ability of the protein to be successfully transported through the membrane.

The presence of internally truncated proteins (ie proteins lacking the amino acids encoded by one or more exons) is preferable. If the CYPD protein is knocked out, there may be problems with elevation of PPIF gene transcription as the body tries to compensate for the reduction in the total amount of CYPD protein. In contrast, the presence of an internally truncated protein (preferably lacking one or more of the features of the complete CYPD protein), should be sufficient to prevent elevated transcription, but still provide a therapeutic advantage due to a reduction in the total amount of functional CYPD protein.

The antisense oligomer induced exon skipping of the present invention need not completely or even substantially ablate the function of the CYPD protein. Preferably, the exon skipping process results in a reduced or compromised functionality of the CYPD protein.

The skipping process of the present invention, using antisense oligomers, may skip an individual exon, or may result in skipping two or more exons at once.

The antisense oligomers of the invention may be a combination of two or more antisense oligomers capable of binding to a selected target to induce exon exclusion in a PPIF gene transcript. The combination may be a cocktail of two or more antisense oligomers and/or a construct comprising two or more antisense oligomers joined together.

TABLE 3
List of antisense oligonucleotide sequences used in this study

| | | | |
|---|---|---|---|
| SEQ ID NO | SITE | NAME | RNA Antisense Sequence |

Exon 2

| | | | |
|---|---|---|---|
| 1 | CYPD E2D (+1−24) | CDm2.1 | CAGAAGGCCCCAGGCCCUGUCUUACC |
| 2 | CYPD E2D (+12−13) | CDm2.2 | GGCCCUGUCUUACCAGCUGUCUUUG |
| 3 | CYPD E2A (+6+30) | CDm2.3 | AGCUGUCUUUGGGACGACAUCUGCC |
| 4 | CYPD E2A (+20+5) | CDm2.4 | UUCAGCUGUUGGGGGAGAAGAAGGA |
| 5 | CYPD E2A (+4+28) | CDm2.5 | CUGUCUUUGGGACGACAUCUGCCUU |
| 6 | CYPD E2A (−24+1) | CDm2.6 | GCUGUUGGGGGAGAAGAAGGACAAAA |

Exon 3

| | | |
|---|---|---|
| 7 | CDm3.1 | CUUACAGAAGAGGAAACUACAUUACC |
| 8 | CDm3.2 | CUGGCACAUGAAGGAAGGGAUCACC |
| 9 | CDm3.3 | CUGUGGAAGGUGGAGCCUUUGUAG |
| 10 | CDm3.4 | CCGAAGCCCUUCUCACCAGUGCAC |
| 11 | CDm3.5 | AGGGCUCUGAAGUUCUCUGAGGGUC |
| 12 | CDm3.6 | UCUGAGGGUCGAAGAGAAACACAGGU |
| 13 | CDm3.7 | CCAGUGCACAGGGCUCUGAAGUUCU |
| 14 | CDm3.41 | AAGCCCUUCUCACCAGUGCACAGG |
| 15 | CDm3.42 | CCCUUCUCACCAGUGCACAGGGCU |
| 16 | CDm3.43 | UAGCCGAAGCCCUUCUCACCAGUG |
| 17 | CDm3.44 | UUUGUAGCCGAAGCCCUUCUCACCA |
| 18 | CDm3.45 | CCUUUGUAGCCGAAGCCCUUCUCA |

Exon 4

| | | |
|---|---|---|
| 19 | CDm4.1 | CACCCUAGAGGAGGCCCCCACUCACC |
| 20 | CDm4.2 | CUGGCCCCACGUGCUUCAGUGUAAA |
| 21 | CDm4.3 | GUUCUCGUCAGGAAAGCGGCUUCCG |
| 22 | CDm4.4 | UAGAUGGACUUCCCGCCUGUGCCAU |
| 23 | CDm4.5 | UGUGGUUGGUGAAGUCGCCCGCCUG |
| 24 | CDm4.6 | CCUGUGAGCACAAAGCACCUUCUGAG |
| 25 | CDm4.7 | CAUUGUGGUUGGUGAAGUCGCCCGC |
| 26 | CDm4.41 | CCGUAGAUGGACUUCCCGCCUGUGC |
| 27 | CDm4.42 | CUUCCGUAGAUGGACUUCCCGCCUG |
| 28 | CDm4.43 | CGGCUUCCGUAGAUGGACUUCCCGC |
| 29 | CDm4.44 | AUGGACUUCCCGCCUGUGCCAUUGU |
| 30 | CDm4.45 | GACUUCCCGCCUGUGCCAUUGUGGU |
| 31 | CDm4.46 | UUCCCGCCUGUGCCAUUGUGGUUGG |

Exon 5

| | | | |
|---|---|---|---|
| 32 | | CDm5.1 | AGAGGGCCUGGGGCAGGGAACUCACC |
| 33 | | CDm5.2 | CAGUCUGUCUUUAUGGUGCAGAUGA |
| 34 | | CDm5.3 | AGAACUGGGAGCCGUUGGUGUUAGG |
| 35 | | CDm5.4 | ACCAGCAUUAGCCAUGGACAGGACAC |
| 36 | | CDm5.5 | CCUGCAAAAACCAGCAGGGCUGAGCC |
| 37 | | CDm5.31 | AGAUGAAGAACUGGGAGCCGUUGGU |
| 38 | | CDm5.32 | ACUGGGAGCCGUUGGUGUUAGGACC |
| 39 | | CDm5.33 | GGGAGCCGUUGGUGUUAGGACCAGC |
| 40 | | CDm5.34 | AGCCGUUGGUGUUAGGACCAGCAUU |
| 41 | | CDm5.35 | CGUUGGUGUUAGGACCAGCAUUAGC |
| 42 | | CDm5.41 | AGGACCAGCAUUAGCCAUGGACAGGA |
| 43 | | CDm5.42 | GUUAGGACCAGCAUUAGCCAUGGACA |
| 44 | | CDm5.43 | GGUGUUAGGACCAGCAUUAGCCAUGG |
| 45 | Scrambled Control | 953 | AUUCCACGACCGCGUUAUGACCGAG |

There is also provided a method for modulating splicing in a PPIF gene transcript, the method including the step of:
a) providing one or more of the antisense oligomers as described herein and allowing the oligomer(s) to bind to a target nucleic acid site.

According to yet another aspect of the invention, there is provided a splice modulation target nucleic acid sequence for PPIF comprising the DNA equivalents of the nucleic acid sequences selected from Table 3 or the group consisting of SEQ ID NOs: 1-44, more preferably SEQ ID NOs: 35 or 37, and sequences complementary thereto.

Designing antisense oligomers to completely mask consensus splice sites may not necessarily generate a change in splicing of the targeted exon. Furthermore, the inventors have discovered that size or length of the antisense oligomer itself is not always a primary factor when designing antisense oligomers. With some targets such as IGTA4 exon 3, antisense oligomers as short as 20 bases were able to induce some exon skipping, in certain cases more efficiently than other longer (eg 25 bases) oligomers directed to the same exon.

The inventors have also discovered that there does not appear to be any standard motif that can be blocked or masked by antisense oligomers to redirect splicing. It has been found that antisense oligomers must be designed, and their individual efficacy evaluated empirically.

More specifically, the antisense oligomer may be selected from those set forth in Table 3. The sequences are preferably selected from the group consisting of any one or more of any one or more of SEQ ID NOs: 1-44, more preferably SEQ ID NOs: 35 or 37 and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a PPIF gene transcript.

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an antisense oligomer need not be 100% complementary to that of its target sequence to be specifically hybridisable. An antisense oligomer is specifically hybridisable when binding of the compound to the target DNA or RNA molecule interferes with the normal function of the target DNA or RNA product, and there is a sufficient degree of complementarity to avoid non-specific binding of the antisense oligomer to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the antisense oligomers of the present invention may include oligomers that selectively hybridise to the sequences provided in Table 3, or SEQ ID NOs: 1-44, more preferably SEQ ID NOs: 35 or 37.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of antisense oligomers may need to be selected by the method of the invention wherein each is directed to a different region responsible for inducing inclusion of the desired exon and/or intron. At a given ionic strength and pH, the Tm is the temperature at which 50% of a target sequence hybridizes to a complementary polynucleotide. Such hybridization may occur with “near” or “substantial” complementarity of the antisense oligomer to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the antisense oligomer. The length of identity comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the antisense oligomer sequences of the invention preferably have at least 75%, more preferably at least 85%, more preferably at least 86, 87, 88, 89 or 90% identity to the sequences shown in the sequence listings herein. More preferably there is at least 91, 92, 93 94, or 95%, more preferably at least 96, 97, 98% or 99%, identity. Generally, the shorter the length of the antisense oligomer, the greater the identity required to obtain selective hybridisation. Consequently, where an antisense oligomer of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the antisense oligomers set out in the sequence listings herein. Nucleotide identity comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The antisense oligomer of the present invention may have regions of reduced identity, and regions of exact identity with the target sequence. It is not necessary for an oligomer to have exact identity for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect identity. Such sequence mismatches will preferably have no or very little loss of splice switching activity. Even more preferably, such sequence mismatches will have increased translation modulating activity

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” or “augment” or “augmenting” refer generally to the ability of one or antisense oligomers or compositions to produce or cause a greater physiological response (i.e., downstream effects) in a cell or a subject relative to the response caused by either no antisense oligomer or a control compound. The terms “decreasing” or “decrease” refer generally to the ability of one or antisense oligomers or compositions to produce or cause a reduced physiological response (i.e., downstream effects) in a cell or a subject relative to the response caused by either no antisense oligomer or a control compound.

Relevant physiological or cellular responses (in vivo or in vitro) will be apparent to persons skilled in the art, and may include increases in the exclusion of specific exons in a PPIF-coding pre-mRNA, decreases in the amount of PPIF-coding pre-mRNA or decreases in the expression of functional CYPD protein in a cell, tissue, or subject in need thereof. An “decreased” or “reduced” amount is typically a statistically significant amount, and may include a decrease that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7. 1.8) less than the amount produced when no antisense oligomer is present (the absence of an agent), or a control compound is used.

The term “reduce” or “inhibit” may relate generally to the ability of one or more antisense oligomers or compositions to “decrease” a relevant physiological or cellular response, such as a symptom of a disease or pathology described herein, as measured according to routine techniques in the diagnostic art. Relevant physiological or cellular responses (in vivo or in vitro) will be apparent to persons skilled in the art, and may include reductions in the symptoms of a disease or pathology such as a disease chosen from the list comprising: ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease.

More preferably, the liver disease is non-alcoholic fatty liver disease (NAFLD) or Non-alcoholic steatohepatitis (NASH).

More preferably the kidney disease is renal inflammation, acute kidney injury, renal fibrosis, diabetic nephropathy.

A “decrease” in a response may be statistically significant as compared to the response produced by no antisense oligomer or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an antisense oligomer may vary, as long as it is capable of binding selectively to the intended location within the pre-mRNA molecule. The length of such sequences can be determined in accordance with selection procedures described herein. Generally, the antisense oligomer will be from about 10 nucleotides in length, up to about 50 nucleotides in length. It will be appreciated, however, that any length of nucleotides within this range may be used in the method. Preferably, the length of the antisense oligomer is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “antisense oligomer” refers to a linear sequence of nucleotides, or nucleotide analogs, that allows the nucleobase to hybridize to a target sequence in an RNA by Watson-Crick base pairing, to form an oligonucleotide: RNA heteroduplex within the target sequence. The terms “antisense oligomer”, “antisense oligonucleotide”, “oligomer” and “antisense compound” may be used interchangeably to refer to an oligonucleotide. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), and 2′-O-Methyl oligonucleotides, among other antisense agents known in the art.

Included are non-naturally-occurring antisense oligomers, or “oligonucleotide analogs”, including antisense oligomers or oligonucleotides having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing antisense oligomers is the methylation of the 2′ hydroxyribose position and the incorporation of a phosphorothioate backbone produces molecules that superficially resemble RNA but that are much more resistant to nuclease degradation, although persons skilled in the art of the invention will be aware of other forms of suitable backbones that may be useable in the objectives of the invention.

To avoid degradation of pre-mRNA and/or mRNA during duplex formation with the antisense oligomers, the antisense oligomers used in the method may be adapted to minimise or prevent cleavage by endogenous RNase H. This property is highly preferred, as the treatment of the RNA with the unmethylated oligomers, either intracellular or in crude extracts that contain RNase H, leads to degradation of the pre-mRNA: antisense and/or mRNA: antisense oligomer duplexes. Any form of modified antisense oligomers that is capable of by-passing or not inducing such degradation may be used in the present method. The nuclease resistance may be achieved by modifying the antisense oligomers of the invention so that it comprises partially unsaturated aliphatic hydrocarbon chain and one or more polar or charged groups including carboxylic acid groups, ester groups, and alcohol groups.

Antisense oligomers that do not activate RNase H can be made in accordance with known techniques (see, e.g., U.S. Pat. No. 5,149,797). Such antisense oligomers, which may be deoxyribonucleotide or ribonucleotide sequences, simply contain any structural modification which sterically hinders or prevents binding of RNase H to a duplex molecule containing the oligomer as one member thereof, which structural modification does not substantially hinder or disrupt duplex formation. Because the portions of the oligomer involved in duplex formation are substantially different from those portions involved in RNase H binding thereto, numerous antisense oligomers that do not activate RNase H are available. For example, such antisense oligomers may be oligomers wherein at least one, or all, of the inter-nucleotide bridging phosphate residues are modified phosphates, such as methyl phosphonates, methyl phosphorothioates, phosphoromorpholidates, phosphoropiperazidates boranophosphates, amide linkages and phosphoramidates. For example, every other one of the internucleotide bridging phosphate residues may be modified as described. In another non-limiting example, such antisense oligomers are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

An example of antisense oligomers which when duplexed with RNA are not cleaved by cellular RNase H is 2′-O-methyl derivatives. Such 2′-O-methyl-oligoribonucleotides are stable in a cellular environment and in animal tissues, and their duplexes with RNA have higher Tm values than their ribo- or deoxyribo-counterparts. Alternatively, the nuclease resistant antisense oligomers of the invention may have at least one of the last 3′-terminus nucleotides fluoridated. Still alternatively, the nuclease resistant antisense oligomers of the invention have phosphorothioate bonds linking between at least two of the last 3-terminus nucleotide bases, preferably having phosphorothioate bonds linking between the last four 3′-terminal nucleotide bases.

Increased splice-switching may also be achieved with alternative oligonucleotide chemistry. For example, the antisense oligomer may be chosen from the list comprising: phosphoramidate or phosphorodiamidate morpholino oligomer (PMO, PMO-X, PPMO); peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′O-Methyl-modified oligomer (2′-OMe); 2′-O-methoxy ethyl (2′-MOE); 2′-fluoro (2′F), 2′-fluroarabino (FANA); unlocked nucleic acid (UNA); hexitol nucleic acid (HNA); cyclohexenyl nucleic acid (CeNA); 2′-amino (2′—NH2); 2′-O-ethyleneamine or any combination of the foregoing as mixmers or as gapmers. To further improve the delivery efficacy, the above-mentioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct exon skipping antisense oligomers. Antisense oligomer-induced splice modification of the human PPIF gene transcripts have generally used either oligoribonucleotides, PNAs, 2OMe or MOE modified bases on a phosphorothioate backbone. Although 2OMeAOs are used for oligo design, due to their efficient uptake in vitro when delivered as cationic lipoplexes, these compounds are susceptible to nuclease degradation and are not considered ideal for in vivo or clinical applications. When alternative chemistries are used to generate the antisense oligomers of the present invention, the uracil (U) of the sequences provided herein may be replaced by a thymine (T).

While the antisense oligomers described above are a preferred form of the antisense oligomers of the present invention, the present invention includes other oligomeric antisense molecules, including but not limited to oligomer mimetics such as are described below.

Specific examples of preferred antisense oligomers useful in this invention include oligomers containing modified backbones or non-natural inter-nucleoside linkages. As defined in this specification, oligomers having modified backbones include those that retain a phosphorus atom in the backbone and those that do not have a phosphorus atom in the backbone. For the purposes of this specification, and as sometimes referenced in the art, modified oligomers that do not have a phosphorus atom in their inter-nucleoside backbone can also be considered to be antisense oligomers.

In other preferred oligomer mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligomer mimetic that has been shown to have excellent hybridization properties, is referred to as a peptide nucleic acid (PNA). In PNA compounds, the sugar-backbone of an oligomer is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligomer (PMO) oligomeric compounds, which are not degraded by any known nuclease or protease. These compounds are uncharged, do not activate RNase H activity when bound to a RNA strand.

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C., even more particularly when combined with 2′-O-methoxyethyl sugar modifications.

Another modification of the oligomers of the invention involves chemically linking to the oligomer one or more moieties or conjugates that enhance the activity, cellular distribution or cellular uptake of the oligomer. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, myristyl, or an octadecylamine or hexylamino-carbonyl-oxycholesterol moiety.

Cell penetrating peptides have been added to phosphorodiamidate morpholino oligomers to enhance cellular uptake and nuclear localization. Different peptide tags have been shown to influence efficiency of uptake and target tissue specificity, as shown in Jearawiriyapaisarn et al. (2008), Mol. Ther. 16 9, 1624-1629.

It is not necessary for all positions in a given compound to be uniformly modified, and in fact more than one of the aforementioned modifications may be incorporated in a single compound or even at a single nucleoside within an oligomer. The present invention also includes antisense oligomers that are chimeric compounds. “Chimeric” antisense oligomers or “chimeras,” in the context of this invention, are antisense oligomers, particularly oligomers, which contain two or more chemically distinct regions, each made up of at least one monomer unit, i.e., a nucleotide in the case of an oligomer compound. These oligomers typically contain at least one region wherein the oligomer is modified so as to confer upon the oligomer or antisense oligomer increased resistance to nuclease degradation, increased cellular uptake, and an additional region for increased binding affinity for the target nucleic acid.

The activity of antisense oligomers and variants thereof can be assayed according to routine techniques in the art. The expression levels of surveyed RNAs and proteins may be assessed by any of a wide variety of well-known methods for detecting expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays. Protein expression levels can be assessed by western blot and/or ELISA assays from a cell, tissue or organism, and by assessing downstream functional or physiological effects.

If two or more different sized transcripts are present, the resulting proteins may be assessed by any of a wide variety of well-known methods for detecting the expression of the relevant protein. Non-limiting examples of such methods include immunological methods for detection of proteins; protein purification methods; mass spectrometry; and protein function or activity assays.

The present invention provides antisense oligomer induced splice-switching of the PPIF gene transcript, clinically relevant oligomer chemistries and delivery systems to direct PPIF splice modulation to therapeutic levels. Clinically relevant decreases in the amount of full length PPIF mRNA, and hence CYPD protein from PPIF gene transcription, are achieved by:
1) oligomer refinement in vitro using fibroblast cell lines, through experimental assessment of (i) intronic-enhancer target motifs, (ii) antisense oligomer length and development of oligomer cocktails, (iii) choice of chemistry, and (iv) the addition of cell-penetrating peptides (CPP) to enhance oligomer delivery; and
2) detailed evaluation of a novel approach to generate PPIF transcripts with one or more missing exons.

As such, it is demonstrated herein that processing of PPIF pre-mRNA can be modulated with specific antisense oligomers. In this way functionally significant decreases in the amount of CYPD protein can be obtained, thereby reducing the severe disease or pathology associated with PPIF gene expression, including: ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease.

The antisense oligomers used in accordance with this invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The antisense oligomers of the invention are synthesised in vitro and do not include antisense compositions of biological origin, or genetic vector constructs designed to direct the in vivo synthesis of antisense oligomers. The molecules of the invention may also be mixed, encapsulated, conjugated or otherwise associated with other molecules, molecule structures or mixtures of compounds, as for example, liposomes, receptor targeted molecules etc.

The antisense oligomers may be formulated for oral, topical, parenteral or other delivery, particularly formulations for injectable delivery. The formulations may be formulated for assisting in uptake, distribution and/or absorption at the site of delivery or activity.

### Method of Treatment

According to a still further aspect of the invention, there is provided one or more antisense oligomers as described herein for use in an antisense oligomer-based therapy. Preferably, the therapy is for a disease or pathology related to PPIF gene expression. More preferably, the therapy for a disease or pathology related to PPIF gene expression is therapy for a disease or pathology chosen from the list in Table 1: ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease.

More preferably, the liver disease is non-alcoholic fatty liver disease (NAFLD) or Non-alcoholic steatohepatitis (NASH).

More preferably, kidney disease is renal inflammation, acute kidney injury, renal fibrosis and diabetic nephropathy.

Most preferably the disease associated with PPIF gene expression in a patient is a liver disease chosen from: alcoholic liver disease (ALD), autoimmune hepatitis, non-alcoholic fatty liver disease (NAFLD), Non-alcoholic steatohepatitis (NASH), hepatitis C, hepatitis B or hepatocellular carcinoma.

More specifically, the antisense oligomer may be selected from Table 3, or the group consisting of any one or more of SEQ ID NOs: 1-44, more preferably SEQ ID NOs: 35 or 37, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a PPIF gene transcript.

The invention extends also to a combination of two or more antisense oligomers capable of binding to a selected target to induce exon exclusion in a PPIF gene transcript. The combination may be a cocktail of two or more antisense oligomers, a construct comprising two or more antisense oligomers joined together for use in an antisense oligomer-based therapy.

There is therefore provided a method to treat, prevent or ameliorate the effects of a disease or pathology associated with PPIF gene expression, comprising the step of:
a) administering to the patient an effective amount of one or more antisense oligomers or pharmaceutical composition comprising one or more antisense oligomers as described herein.

Preferably the disease or pathology associated with PPIF gene expression in a patient is chosen from the list comprising: ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease. More preferably the disease or pathology is a liver disease chosen from: non-alcoholic fatty liver disease (NAFLD), Non-alcoholic steatohepatitis (NASH), or hepatocellular carcinoma.

Therefore, the invention provides a method to treat, prevent or ameliorate the effects of a disease or pathology associated with PPIF gene expression, comprising the step of:
a) administering to the patient an effective amount of one or more antisense oligomers or pharmaceutical composition comprising one or more antisense oligomers as described herein.

Preferably, the therapy is used to reduce the levels of functional CYPD protein via an exon skipping strategy. The reduction in levels of CYPD is preferably achieved by reducing the transcripts level through modifying pre-mRNA splicing in the PPIF gene transcript or part thereof.

The reduction in PPIF will preferably lead to a reduction in the quantity, duration or severity of the symptoms of a PPIF-related disease or pathology, such as a disease or pathology associated with PPIF gene expression chosen from the list comprising: ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or pathology being treated, delaying the onset of that disease or pathology, or reducing the severity of its onset. “Treatment” or “prophylaxis” does not necessarily indicate complete eradication, cure, or prevention of the disease or pathology, or associated symptoms thereof.

The subject with the disease or pathology associated with PPIF gene expression may be a mammal, including a human.

The antisense oligomers of the present invention may also be used in conjunction with alternative therapies, such as drug therapies.

The present invention therefore provides a method of treating, preventing or ameliorating the effects of a disease or pathology associated with PPIF gene expression, wherein the antisense oligomers of the present invention and administered sequentially or concurrently with another alternative therapy associated with treating, preventing or ameliorating the effects of a disease or pathology associated with PPIF gene expression. Preferably, the disease or pathology is chosen from the list comprising: ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease.

### Delivery

The antisense oligomers of the present invention also can be used as a prophylactic or therapeutic, which may be utilised for the purpose of treatment of a disease or pathology associated with PPIF gene expression. Accordingly, in one embodiment the present invention provides antisense oligomers that bind to a selected target in the PPIF pre-mRNA to induce efficient and consistent exon skipping as described herein, in a therapeutically effective amount, admixed with a pharmaceutically acceptable carrier, diluent, or excipient.

There is also provided a pharmaceutical, prophylactic, or therapeutic composition to treat, prevent or ameliorate the effects of a disease or pathology related to PPIF gene expression in a patient, the composition comprising:
a) one or more antisense oligomers as described herein and
b) one or more pharmaceutically acceptable carriers and/or diluents.

The antisense oligomer may be administered at regular intervals for a short time period, e.g., daily for two weeks or less. However, in many cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

Dosing may be dependent on severity and responsiveness of the disease state to be treated, with the course of treatment lasting from several days to several months, or until a cure is effected or a diminution of the disease state is achieved. Alternatively, dosing may be titrated against disease progression rate. A baseline progression is established. Then the progression rate after an initial once off dose is monitored to check that there is a reduction in the rate. Preferably, there is no progression after dosing. Preferably, re-dosing is only necessary if progression rate is unchanged. Successful treatment preferably results in no further progression of the disease or even some recovery of vision. Optimal dosing schedules can be calculated from measurements of drug accumulation in the body of the patient. Persons of ordinary skill can easily determine optimum dosages, dosing methodologies and repetition rates.

Optimum dosages may vary depending on the relative potency of individual oligomers and can generally be estimated based on EC50s found to be effective in in vitro and in vivo animal models.

Repetition rates for dosing depend on progression rate of the disease or pathology. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state.

An effective in vivo treatment regimen using the antisense oligomers of the invention may vary according to the duration, dose, frequency and route of administration, as well as the condition of the subject under treatment (i.e., prophylactic administration versus administration in response to localized or systemic infection). Accordingly, such in vivo therapy will often require monitoring by tests appropriate to the particular type of disorder under treatment, and corresponding adjustments in the dose or treatment regimen, in order to achieve an optimal therapeutic outcome.

Treatment may be monitored, e.g., by general indicators of disease known in the art. The efficacy of an in vivo administered antisense oligomers of the invention may be determined from biological samples (tissue, blood, urine etc.) taken from a subject prior to, during and subsequent to administration of the antisense oligomer. Assays of such samples include (1) monitoring the presence or absence of heteroduplex formation with target and non-target sequences, using procedures known to those skilled in the art, e.g., an electrophoretic gel mobility assay; (2) monitoring the amount of a mutant mRNA in relation to a reference normal mRNA or protein as determined by standard techniques such as RT-PCR, Northern blotting, ELISA or Western blotting.

Intranuclear oligomer delivery is a major challenge for antisense oligomers. Different cell-penetrating peptides (CPP) localize antisense oligomers to varying degrees in different conditions and cell lines, and novel CPPs have been evaluated by the inventors for their ability to deliver antisense oligomers to the target cells. The terms CPP or “a peptide moiety which enhances cellular uptake” are used interchangeably and refer to cationic cell penetrating peptides, also called “transport peptides”, “carrier peptides”, or “peptide transduction domains”. The peptides, as shown herein, have the capability of inducing cell penetration within about or at least about 30%, 40%, 50%, 60%, 70%, 80%, 90%, or 100% of cells of a given cell culture population and allow macromolecular translocation within multiple tissues in vivo upon systemic administration. CPPs are well-known in the art and are disclosed, for example in U.S. Application No. 2010/0016215, which is incorporated by reference in its entirety.

The present invention therefore provides antisense oligomers of the present invention win combination with cell-penetrating peptides for manufacturing therapeutic pharmaceutical compositions.

### Excipients

The antisense oligomers of the present invention are preferably delivered in a pharmaceutically acceptable composition. The composition may comprise about 1 nM to 1000 nM of each of the desired antisense oligomer(s) of the invention. Preferably, the composition may comprise about 1 nM to 500 nM, 10 nM to 500 nM, 50 nM to 750 nM, 10 nM to 500 nM, 1 nM to 100 nM, 1 nM to 50 nM, 1 nM to 40 nM, 1 nM to 30 nM, 1 nM to 20 nM, most preferably between 1 nM and 10 nM of each of the antisense oligomer(s) of the invention.

The composition may comprise about 1 nm, 2 nm, 3 nm, 4 nm, 5 nm, 6 nm, 7 nm, 8 nm, 9 nm, 10 nm, 20 nm, 50 nm, 75 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm or 1000 nm of each of the desired antisense oligomer(s) of the invention.

The present invention further provides one or more antisense oligomers adapted to aid in the prophylactic or therapeutic treatment, prevention or amelioration of symptoms of a disease such as an PPIF gene expression related disease or pathology in a form suitable for delivery to a patient.

The phrase “pharmaceutically acceptable” refers to molecular entities and compositions that are physiologically tolerable and do not typically produce an allergic or similarly untoward reaction, such as gastric upset and the like, when administered to a patient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Remington: The Science and Practice of Pharmacy, 22nd Ed., Pharmaceutical Press, PA (2013).

In a more specific form of the invention there are provided pharmaceutical compositions comprising therapeutically effective amounts of one or more antisense oligomers of the invention together with pharmaceutically acceptable diluents, preservatives, solubilizers, emulsifiers, adjuvants, and/or carriers. Such compositions include diluents of various buffer content (e.g. Tris-HCl, acetate, phosphate), pH and ionic strength and additives such as detergents and solubilizing agents (e.g. Tween 80, Polysorbate 80), anti-oxidants (e.g., ascorbic acid, sodium metabisulfite), preservatives (e.g. Thimersol, benzyl alcohol) and bulking substances (e.g., lactose, mannitol). The material may be incorporated into particulate preparations of polymeric compounds such as polylactic acid, polyglycolic acid, etc. or into liposomes. Hyaluronic acid may also be used. Such compositions may influence the physical state, stability, rate of in vivo release, and rate of in vivo clearance of the present proteins and derivatives. See, for example, Remington: The Science and Practice of Pharmacy, 22nd Ed., Pharmaceutical Press, PA (2013). The compositions may be prepared in liquid form, or may be in dried powder, such as a lyophilised form.

It will be appreciated that pharmaceutical compositions provided according to the present invention may be administered by any means known in the art. The pharmaceutical compositions for administration are administered by injection, orally, topically or by the pulmonary or nasal route. For example, the antisense oligomers may be delivered by topical, intravenous, intra-arterial, intraperitoneal, intramuscular or subcutaneous routes of administration. The appropriate route may be determined by one of skill in the art, as appropriate to the condition of the subject under treatment.

Formulations for topical administration include those in which the oligomers of the disclosure are in admixture with a topical delivery agent such as lipids, liposomes, fatty acids, fatty acid esters, steroids, chelating agents and surfactants. Lipids and liposomes include neutral (e.g. dioleoylphosphatidyl DOPE ethanolamine, dimyristoylphosphatidyl choline DMPC, disteroylphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). For topical or other administration, oligomers of the disclosure may be encapsulated within liposomes or may form complexes thereto, in particular to cationic liposomes. Alternatively, oligomers may be complexed to lipids, in particular to cationic lipids. Fatty acids and esters, pharmaceutically acceptable salts thereof, and their uses are further described in U.S. Pat. No. 6,287,860 and/or U.S. patent application Ser. No. 09/315,298 filed on May 20, 1999.

In certain embodiments, the antisense oligomers of the disclosure can be delivered by topical or transdermal methods (e.g., via incorporation of the antisense oligomers into, e.g., emulsions, with such antisense oligomers optionally packaged into liposomes). Such topical or transdermal and emulsion/liposome-mediated methods of delivery are described for delivery of antisense oligomers in the art, e.g., in U.S. Pat. No. 6,965,025.

The antisense oligomers described herein may also be delivered via an implantable device. Design of such a device is an art-recognized process, with, e.g., synthetic implant design described in, e.g., U.S. Pat. No. 6,969,400.

Compositions and formulations for administration, including injection, topical delivery and implants may include sterile aqueous solutions which may also contain buffers, diluents and other suitable additives such as, but not limited to, penetration enhancers, carrier compounds and other pharmaceutically acceptable carriers or excipients.

The delivery of a therapeutically useful amount of antisense oligomers may be achieved by methods previously published. For example, delivery of the antisense oligomer may be via a composition comprising an admixture of the antisense oligomer and an effective amount of a block copolymer. An example of this method is described in US patent application US20040248833. Other methods of delivery of antisense oligomers to the nucleus are described in Mann C J et al. (2001) Proc, Natl. Acad. Science, 98 (1) 42-47, and in Gebski et al. (2003) Human Molecular Genetics, 12 (15): 1801-1811. A method for introducing a nucleic acid molecule into a cell by way of an expression vector either as naked DNA or complexed to lipid carriers, is described in U.S. Pat. No. 6,806,084.

Antisense oligomers can be introduced into cells using art-recognized techniques (e.g., transfection, electroporation, fusion, liposomes, colloidal polymeric particles and viral and non-viral vectors as well as other means known in the art). The method of delivery selected will depend at least on the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

It may be desirable to deliver the antisense oligomer in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. These colloidal dispersion systems can be used in the manufacture of therapeutic pharmaceutical compositions.

Liposomes are artificial membrane vesicles, which are useful as delivery vehicles in vitro and in vivo. These formulations may have net cationic, anionic, or neutral charge characteristics and have useful characteristics for in vitro, in vivo and ex vivo delivery methods. It has been shown that large unilamellar vesicles can encapsulate a substantial percentage of an aqueous buffer containing large macromolecules. RNA and DNA can be encapsulated within the aqueous interior and be delivered to cells in a biologically active form (Fraley, et al., Trends Biochem. Sci. 6:77, 1981).

In order for a liposome to be an efficient gene transfer vehicle, the following characteristics should be present: (1) encapsulation of the antisense oligomer of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988). The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

As known in the art, antisense oligomers may be delivered using, for example, methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The antisense oligomer may also be combined with other pharmaceutically acceptable carriers or diluents to produce a pharmaceutical composition. Suitable carriers and diluents include isotonic saline solutions, for example phosphate-buffered saline. The composition may be formulated for topical, parenteral, intramuscular, intravenous, subcutaneous, intraocular, oral, or transdermal administration.

The routes of administration described are intended only as a guide since a skilled practitioner will be able to readily determine the optimum route of administration and any dosage for any particular subject and disease or pathology.

Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51 (18), suppl.: 5074S-5079S); integration into non-retrovirus vectors (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. Examples of review articles of human gene therapy procedures are Anderson, Science (1992) 256:808-813; Barteau et al. (2008), Curr Gene Ther; 8 (5): 313-23; Mueller et al. (2008). Clin Rev Allergy Immunol; 35 (3): 164-78; Li et al. (2006) Gene Ther., 13 (18): 1313-9; Simoes et al. (2005) Expert Opin Drug Deliv; 2 (2): 237-54.

The antisense oligomers of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e. salts that retain the desired biological activity of the parent compound and do not impart undesired toxicological effects thereto. For oligomers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be via topical (including ophthalmic and mucous membranes, as well as rectal delivery), pulmonary (e.g., by inhalation or insufflation of powders or aerosols, including by nebulizer, intratracheal, intranasal, epidermal and transdermal), oral or parenteral routes. Parenteral administration includes intravenous, intra-arterial, subcutaneous, intraperitoneal, intraocular or intramuscular injection or infusion; or intracranial, e.g., intrathecal or intraventricular, administration. Oligomers with at least one 2′-O-methoxyethyl modification are believed to be particularly useful for administration.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

### Use

According to another aspect of the invention there is provided the use of one or more antisense oligomers as described herein in the manufacture of a medicament for the modulation or control of a disease or pathology associated with PPIF gene expression.

The invention also provides for the use of purified and isolated antisense oligomers as described herein, for the manufacture of a medicament to treat, prevent or ameliorate a disease or pathology associated with PPIF gene expression.

There is also provided the use of purified and isolated antisense oligomers as described herein, for the manufacture of a medicament to treat, prevent or ameliorate the effects of a disease or pathology associated with PPIF gene expression.

There is also provided the use of one or more antisense oligomers as described herein for the modulation or control of a disease or pathology associated with PPIF gene expression.

The invention also provides for the use of purified and isolated antisense oligomers as described herein to treat, prevent or ameliorate a disease or pathology associated with PPIF gene expression.

There is also provided the use of purified and isolated antisense oligomers as described herein to treat, prevent or ameliorate the effects of a disease or pathology associated with PPIF gene expression.

Preferably, the PPIF gene expression related disease or pathology is chosen from the list comprising: ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease.

The invention extends, according to a still further aspect thereof, to cDNA or cloned copies of the antisense oligomer sequences of the invention, as well as to vectors containing the antisense oligomer sequences of the invention. The invention extends further also to cells containing such sequences and/or vectors.

### Kits

There is also provided a kit to treat, prevent or ameliorate the effects of a disease or pathology associated with PPIF gene expression in a patient, which kit comprises at least an antisense oligomer as described herein and combinations or cocktails thereof, packaged in a suitable container, together with instructions for its use.

In a preferred embodiment, the kits will contain at least one antisense oligomer as described herein or as shown in Table 3, or SEQ ID NOs: 1-44, more preferably SEQ ID NOs: 35 or 37 or a cocktail of antisense oligomers, as described herein. The kits may also contain peripheral reagents such as buffers, stabilizers, etc.

There is therefore provided a kit to treat, prevent or ameliorate a disease or pathology associated with PPIF gene expression in a patient, which kit comprises at least an antisense oligomer described herein or as shown in Table 3 and combinations or cocktails thereof, packaged in a suitable container, together with instructions for its use.

There is also provided a kit to treat, prevent or ameliorate a disease or pathology associated with PPIF expression in a patient, which kit comprises at least an antisense oligomer selected from the group consisting of any one or more of SEQ ID NOs: 1-44, more preferably SEQ ID NOs: 35 or 37, and combinations or cocktails thereof, packaged in a suitable container, together with instructions for its use.

Preferably, the disease or pathology is chosen from the list comprising: ischaemia reperfusion related injury, oxidative related injury, inflammatory related injury and trauma related injury (affecting the liver, brain, heart, lung, pancreas and kidney), neurodegeneration (Alzheimer's, Parkinson's disease, motor neuron disease), diabetes, metabolic disease (NAFLD/NASH and obesity), skeletal muscle disease, and inflammatory disease. More preferably the disease or pathology is a liver disease chosen from: non-alcoholic fatty liver disease (NAFLD), Non-alcoholic steatohepatitis (NASH), or hepatocellular carcinoma.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the subject such as the lungs, injected into subject, or even applied to and mixed with the other components of the kit.

In an embodiment, the kit of the present invention comprises a composition comprising a therapeutically effective amount of an antisense oligomer capable of binding to a selected target on a PPIF gene transcript to modify pre-mRNA splicing in a PPIF gene transcript or part thereof. In an alternative embodiment, the formulation is in pre-measured, pre-mixed and/or pre-packaged.

The kit of the present invention may also include instructions designed to facilitate user compliance. Instructions, as used herein, refers to any label, insert, etc., and may be positioned on one or more surfaces of the packaging material, or the instructions may be provided on a separate sheet, or any combination thereof. For example, in an embodiment, the kit of the present invention comprises instructions for administering the formulations of the present invention. In one embodiment, the instructions indicate that the formulation of the present invention is suitable for the treatment of a disease or pathology associated with PPIF gene expression. Such instructions may also include instructions on dosage, as well as instructions for administration.

The antisense oligomers and suitable excipients can be packaged individually so to allow a practitioner or user to formulate the components into a pharmaceutically acceptable composition as needed. Alternatively, the antisense oligomers and suitable excipients can be packaged together, thereby requiring de minimis formulation by the practitioner or user. In any event, the packaging should maintain chemical, physical, and aesthetic integrity of the active ingredients.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent or may encompass two or more active agents.

Sequence identity numbers (“SEQ ID NO:”) containing nucleotide and amino acid sequence information included in this specification are collected at the end of the description and have been prepared using the program PatentIn Version 3.0. Each nucleotide or amino acid sequence is identified in the sequence listing by the numeric indicator <210> followed by the sequence identifier (e.g. <210>1, <210>2, etc.). The length, type of sequence and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (e.g. <400>1, <400>2, etc.).

An antisense oligomer nomenclature system was proposed and published to distinguish between the different antisense oligomers (see Mann et al., (2002) J Gen Med 4, 644-654). This nomenclature became especially relevant when testing several slightly different antisense oligomers, all directed at the same target region, as shown below:

$$H \# A / D ( x : y )$$

the first letter designates the species (e.g. H: human, M: murine)
“#” designates target exon number
“A/D” indicates acceptor or donor splice site at the beginning and end of the exon, respectively
(x y) represents the annealing coordinates where “−” or “+” indicate intronic or exonic sequences respectively. As an example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2-18) where the last 2 exonic bases and the first 18 intronic bases correspond to the annealing site of the antisense oligomer. Entirely exonic annealing coordinates that would be represented by A(+65+85), that is the site between the 65th and 85th nucleotide, inclusive, from the start of that exon.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

### Example 1

### Antisense Oligomer (ASON) Mediated Exon Skipping to Induce a Frameshift in PPIF

TABLE 7
Materials

| Reagent | Supplier/Composition |
|---|---|
| OPTIMEM | Gibco_Thermofisher |
| Lipofectamine | Invitrogen_Thermofisher |
| RNAiMax | |
| Oligo (dT)₁₅ 500 ug/ml | Promega |
| M-MLV RT RNase (H-) | Promega |
| PointMutant | |
| 5 × M-MLV RT Buffer | Promega |
| dNTP Mix, 10 mM | Invitrogen |
| GoTaq(R) G2 | Promega |
| Colorless Master Mix | |
| Dulbecco's Modified | DMEM with high glucose, GluMAX TM, (GIBCO), HEPES, phenol red and |
| Eagles Medium | supplemented with streptomycin (10 mg/ml) and penicillin (10 units/ml) |
| (DMEM) | |
| Trypsin | TrypLE TM Express (1x), with EDTA (Invitrogen) |
| Bovine serum albumin | 0.0125 g BSA dissolved in 25 ml PBS-Tween 0.1% |
| (BSA) | |
| Tris Glycine SDS buffer | 10x premixed electrophoresis buffer: 25 mM Tris, 192 |
| 1x (TGS 1x) | mM glycine, 0.1% SDS, pH 8.3 (Bio-Rad Laboratories) |
| Towbin Transfer Buffer | To make up 1x TG; 3 g Tris-base, 14.4 g glycine, 200 ml methanol, |
| | adjust pH to 7.6 and make up to 1 L with Mili-Q water |
| Coomassie stain | 1 Coomassie tablet per 600 ml, dissolved in 0/6% Mili-Q water, 30% |
| | methanol and 10% acetic acid |
| Tris Buffered Saline | To make up 10x TBS; 24.2 g Tris-base, 80 g NaCl, adjust pH to 7.6 |
| 10x (TBS 10x) | |
| Tris buffered saline + | To make up 1 L; as for TBS with 1 ml of Tween |
| Tween 0.1% (TBS-T) | |
| 10x Tris/Glycine/SDS | 1 L, 10x premixed electrophoresis buffer, contains 25 mM Tris, 192 mM |
| | glycine, 0.1% SDS, pH 8.3 following dilution to 1x with water |

### Methods

### Cell Culture

All experiments were performed in the cell line HepG2 and Huh-7 cells (CellBank Australia). Cultures were grown in GluMax Dulbecco's Modified Eagles Medium (DMEM; Gibco and supplemented with 10% fetal calf serum [Serana], Penicillin [10 units/ml] and Streptomycin [10 ug/ml] at 37° C. in 5% CO2. Cells were maintained in flasks (T-25) and sub-cultured every 3-4 days at a ratio of 1 to 6. Cells were not used beyond 25 passages.

### Transfection

The day before transfection HepG2 cells, media was removed from a T-25 flask, and the cell monolayer trypsinised (TrypLE™ Express containing EDTA; Invitrogen) for 15 minutes at 37° C. in 5% CO2. Cells were transferred using a sterile transfer pipette to a 5 ml sterile tube and the cells released from clumps by repeated vigorous passage (10×) through an 18-gauge needle. Cells were transferred to a sterile 15 ml conical centrifuge tube and resuspended in fresh DMEM containing serum. After centrifugation at 750 rpm for 2 minutes, media was removed, and the cell pellet was resuspended in 2 ml of fresh DMEM. 10 ul was removed and mixed with an equal volume of trypan blue (0.05%) and the cell concentration determined using a haemocytometer. Cells were diluted in DMEM with serum/antibiotics and plated into 24 well plates at approximately 50,000 cells per well in 500 ul. The following day (approximately 24 hours later) growth media was removed and replaced with 500 ul of OptiMEM and treated with ASON (to the desired final concentration of 1000 nM, 500 nM or 250 nM) was mixed with RNAiMax (Invitrogen) according to the manufacturers' instructions. In brief, ASONs were combined with serum free OptiMEM and RNAiMax (X ul per each 24 well) and incubated for 5 minutes. The ASON/RNAiMax mixture was added to each well in a dropwise manner, mixed gently and incubated for 24 or 48 hours prior to RNA (for RT-PCR) or protein extraction (for Western Blotting). For protein collection and to prevent further cell proliferation, OptiMEM/transfection reagents was replaced with DMEM supplemented 1% FCS and antibiotics after 24 hours.

### RNA Extraction

Media was removed, and total RNA was extracted using either the RNeasy Mini Kit according column method (Qiagen (kit) or the total RNA Favorgen Blood/Cell culture column kit according to the manufacturer's instructions. Total RNA was eluted in sterile water and quantified using a Nanodrop instrument (Invitrogen).

Annealing and Synthesis of Complementary DNA (cDNA)

For the annealing step, total RNA (250-500 ng) was combined with 0.125 ug of Oligo (dT) 15 (Promega) in WFI, incubated at 70° C. for 5 minutes and cooled rapidly by quenching on ice (0-4° C.) for 5 minutes. For first strand synthesis, annealed primer-templates were gently mixed with 5×M-MLV RT Buffer (Promega), 10 mM dNTP mix (Promega) and MMLV Reverse Transcriptase, RNase H Minus, Point Mutant (Promega). Reactions were incubated at 40° C. for 10 minutes followed by 50° C. for 60 minutes. Reactions were inactivated by heating to 70° C. for 15 minutes. Complementary DNA was used as template for PCR amplification or otherwise stored at −20° C.

### Second Strand Synthesis and PCR Amplification

For a reaction volume of 20 ul, 2 ul of cDNA was combined with CYPD primers (Table 4) and sterile water and an equal volume of GO-TAQ clear 2× master mix. The PCR amplification reaction proceeded according to the cycling conditions set out below in Table 5.

TABLE 4
PCR DNA Oligonucleotides

| SEQ | | | |
|---|---|---|---|
| ID | | | |
| NO | Primer Name | Sequence 5′−>3′ | Supplier |
| 46 | PPIF_Fwd 106 | GACCCGTCCTCTTCCTCCT | IDT DNA |
| 47 | PPIF_Rev 622 | AGCTCAACTGGCCACAGTCT | IDT DNA |
| 48 | mtCypD For_323 | GCAGATGTCGTCCCAAAGA | IDT DNA |
| 49 | mtCypD Rev_323 | TTGACGTGACCGAACACAA | IDT DNA |
| 50 | mtCypD F1 | CGTGTACCTGGACGTGGAC | IDT DNA |
| 51 | mtCypD R1 Ex5 | GAGCCGTTGGTGTTAGGAC | IDT DNA |
| 52 | mtCypD R2 Ex4 | GGCTTCCGTAGATGGACTTC | IDT DNA |

1. Based on the PPIF reference sequence (NM_005729)
amplification with primers SEQ ID 46 and 47 produces a
full-length PCR fragment of 517 base pairs (bp) in size.
2. Based on the PPIF reference sequence (NM_005729)
amplification with primers SEQ ID 48 and 49 produces a
full-length PCR fragment of 323 base pairs (bp) in size.
3. Based on the PPIF reference sequence (NM_005729)
amplification with primers SEQ ID 49 and 50 produces a
full-length PCR fragment of 384 base pairs (bp) in size.

TABLE 5
PCR conditions

| | | | |
|---|---|---|---|
| Stage 1 | Stage 2 | Stage 3 | Stage 4 |
| (Annealing) | (Amplification) | (final extension) | (Hold) |

AMPLIFICATION CONDITIONS FOR SEQ IDs 46/47 PRIMER SET

| | | | |
|---|---|---|---|
| Step 1 94° | Step 1. 94° | Step 1. 72° | Step 1. |
| C. × 2 mins | C. × 45 sec | C. × 5 mins | 11° C. hold |
| | Step 2. 50° | | |
| | C. × 45 sec | | |
| | Step 3. 72° | | |
| | C. × 45 sec | | |
| | Cycles 28-35 | | |

AMPLIFICATION CONDITIONS FOR SEQ IDs 48/49 PRIMER SET

| | | | |
|---|---|---|---|
| Step 1 94° | Step 1. 94° | Step 1. 72° | Step 1. |
| C. × 2 mins | C. × 45 sec | C. × 5 mins | 11° C. hold |
| | Step 2. 50° | | |
| | C. × 45 sec | | |
| | Step 3. 72° | | |
| | C. × 45 sec | | |
| | Cycles 28-35 | | |

### Gel Electrophoresis

PCR reactions (20 ul) were mixed with 4 ul of 6× gel loading buffer and electrophoresed in 2-3.5% agarose gels at 90V for 90 minutes, using a 1×TAE or a TBE buffer system. Gels were pre-stained with Syber Safe DNA gel stain (Thermo Fisher) and bands were visualised and imaged using a Chemi-Doc System (Bio-Rad Laboratories). A 100 bp molecular weight ladder (Axygen) was included for band size estimation.

### Sequencing Analysis

To confirm exon skipping following ASON treatment, PCR bands were excised from agarose gels, purified and subjected to DNA sequencing (by the Australian Genome Resource Facility: AGRF or by the State Agricultural Biotechnology Centre).

### Protein Gel Electrophoresis

Proteins were analysed by sodium dodecyl sulphate polyacrylamide gel electrophoresis (SDS-PAGE; TGX stain free system form Bio-Rad, USA) using a Mini-Protean Tetra Cell assembly (Bio-Rad Laboratories). Gels used were 4%-15% gradient Mini-Protean TGX precast gels (Bio-Rad Laboratories). A total volume of 5 ul of protein and sterile DD water was combined with 4.75 ul Laemmli Sample buffer (Bio-Rad Laboratories) and 0.25 ul dithiothreitol (DTT) and heated at 70° C. for 10 minutes. A Precision Plus pre-stained protein ladder (Bio-Rad) was used as a molecular weight standard. Samples (10 ul) and markers were loaded into pre-rinsed gels and run at 200 volts for 45 minutes. Protein bands were visualised using a ChemDoc System (Bio-Rad) fitted with an appropriate filter.

### Western Transfer and Immunoblotting

Proteins were transferred from SDS-PAGE gels onto 0.45 um Low Fluorescence PVDF membranes using a Turbo-Blot Turbo (BioRad-Laboratories) transfer apparatus using Turbo-Blot Turbo transfer buffer (BioRad Laboratories) 20% ethanol. Membranes were washed in Tris-Buffered Saline (TBS: Tris 20 mM; NaCl 200 mM; pH 7.6) and blocked in TBS-T (TBS with 0.1% Tween-20 plus chicken ovalbumin [Sigma; at 1 mg/ml]) for 1 h. Membranes were incubated in primary antibody (mouse monoclonal anti-cyclophilin F at 1:5000) diluted in TBS-T overnight at 4° C. with gentle rocking. Membranes were rinsed 3× with TBS-T and incubated in TBS-T containing secondary antibodies (Goat anti-mouse IgG labelled with HRP enzyme 1:25,000) at room temperature for 2 hours. Membranes were rinsed 3× with TBS-T then washed 3× in TBS a further 5 minutes. Protein bands were detected using ECL reagent Clarity, BioRad) according to the manufacturer's instructions. The blot was imaged using a ChemiDoc-MP system (Bio-Rad) using the chemiluminescent and fluorescence imaging settings. Image Lab (BioRad) imaging software was used to quantify CYPD expression and the stain free gel image was used to normalise for protein loading.

TABLE 6
Western Blotting materials
WESTERN BLOTTING REAGENTS

| | | | |
|---|---|---|---|
| Antibody | Working conc | Source | Supplier |

Primary antibodies

| | | | |
|---|---|---|---|
| Anti-cyclophilin | 1:5,000 | Rabbit | Abcam |
| D (CYPD) ab | | (polyclonal) | |

Secondary Antibodies

| | | | |
|---|---|---|---|
| Goat Anti-rabbit IgG | 1:5,000 | Goat | Bio-Rad |
| StarBright Blue 520 | | (polyclonal) | |
| Anti-GAPDH hFAB | 1:25,000 | Goat | Bio-Rad |
| Rhodamine | | (polyclonal) | |

### Results

Antisense RNA sequences were designed to interact with the full-length pre-mRNA CYPB transcript and interfere with normal splicing so that either exon 3 or exon 4 were deleted from the mature mRNA transcript. Based on the PPIF reference sequence (NM_005729): exon 2 deletion was predicted to induce 6 pre-mature downstream stop codon(s); exon 3 deletion was predicted to induce 4 pre-mature downstream stop codon(s); exon 4 deletion was predicted to induce 4 pre-mature downstream stop codons and exon 5 deletion was predicted to induce 3 pre-mature downstream stop codon(s).

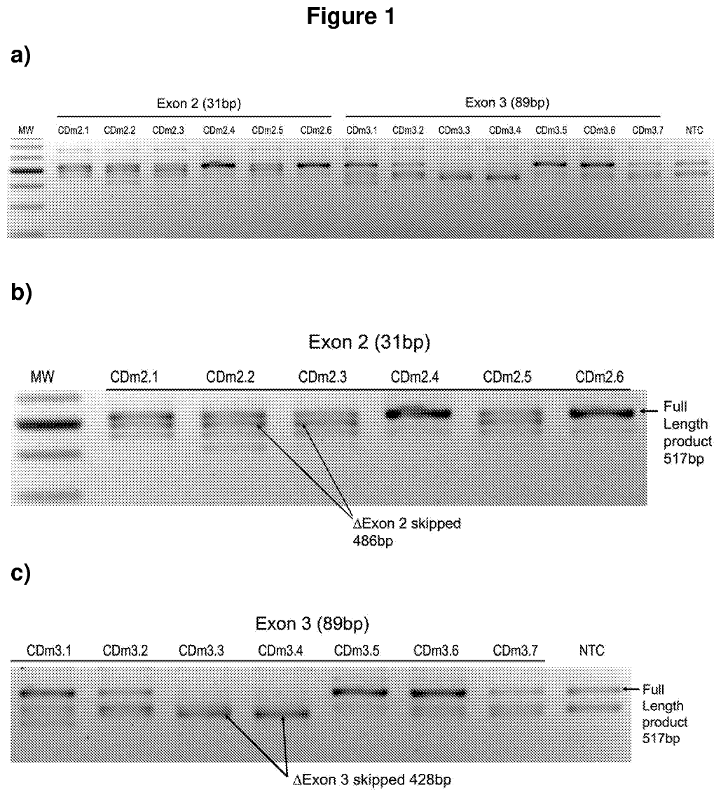

For exon 2 deletion, six (6) ASO's (SEQ IDs1-6) were designed to span either the 5′ prime acceptor site of exon 3, the 3′ donor site of exon 3 and/or sites within exon 2. In these experiments, which were repeated at least once, Hep G2 cells were transfected with 500 nM of each antisense, synthesised in 2′-O-methyl chemistry. Total RNA was collected 24 hours post-transfection and RT-PCR analysis (amplified with primer set SEQ 46/47) was performed to assess the extent of exon-skipping (FIG. 1a). SEQs CDm2.1, 2.2, 2.3 and 2.5 induced comparable levels of exon 2 skipping, and that deletion of exon 2 resulted in a PCR product consistent with the expected size of 31 bp (FIG. 1b).

For exon 3 deletion, seven (7) ASO's (SEQ IDs7-13) were designed to span either the 5′ prime acceptor site of exon 3, the 3′ donor site of exon 3 and/or sites within exon 3. In these experiments, which were repeated at least once, Hep G2 cells were transfected with 500 nM of each antisense, synthesised in 2′-O-methyl chemistry. Total RNA was collected 24 hours post-transfection and RT-PCR analysis (amplified with primer set SEQ 46/47) was performed to assess the extent of exon-skipping (FIG. 1a). SEQs CDm3.3 and CDm3.4 induced comparable levels of exon 4 skipping, and that deletion of exon 4 resulted in a PCR product consistent with the expected size of 89 bp (FIG. 1c).

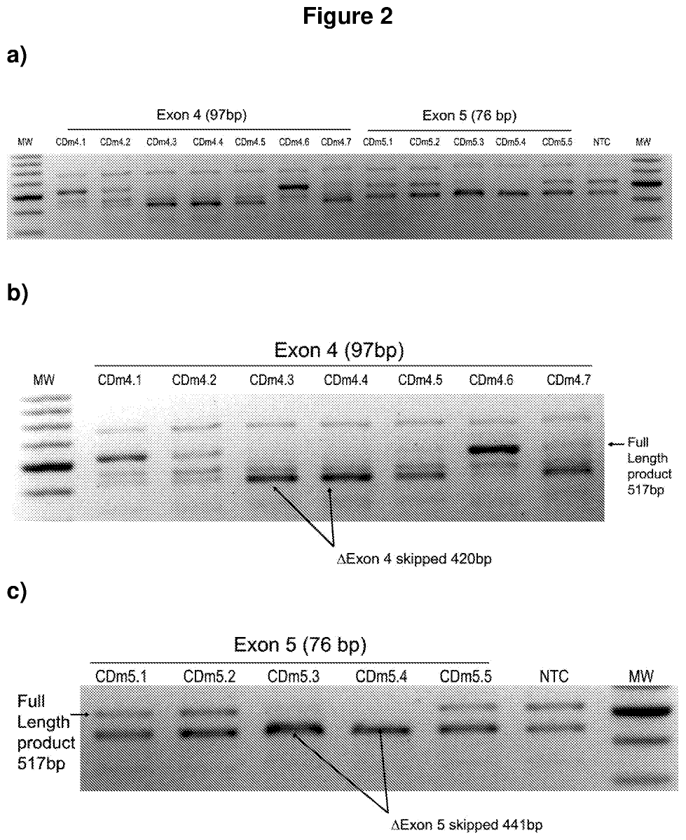

For exon 4 deletion, six (6) ASO's (SEQ IDs19-25) were designed to span either the 5′ prime acceptor site of exon 3, the 3′ donor site of exon 4 and/or sites within exon 4. In these experiments, which were repeated at least once, Hep G2 cells were transfected with 500 nM of each antisense, synthesised in 2′-O-methyl chemistry. Total RNA was collected 24 hours post-transfection and RT-PCR analysis (amplified with primer set SEQ 46/47) was performed to assess the extent of exon-skipping (FIG. 2a). SEQs CDm4.3 and CDm4.4 induced comparable levels of exon 4 skipping, and that deletion of exon 4 resulted in a PCR product consistent with the expected size of 97 bp (FIG. 2b).

For exon 5 deletion, five (5) ASO's (SEQ IDs32-36) were designed to span either the 5′ prime acceptor site of exon 3, the 3′ donor site of exon 5 and/or sites within exon 5. In these experiments, which were repeated at least once, Hep G2 cells were transfected with 500 nM of each antisense, synthesised in 2′-O-methyl chemistry. Total RNA was collected 24 hours post-transfection and RT-PCR analysis (amplified with primer set SEQ 46/47) was performed to assess the extent of exon-skipping (FIG. 2b). SEQs CDm5.3 and CDm5.4 induced comparable levels of exon 5 skipping, and that deletion of exon 5 resulted in a PCR product consistent with the expected size of 76 bp (FIG. 2c).

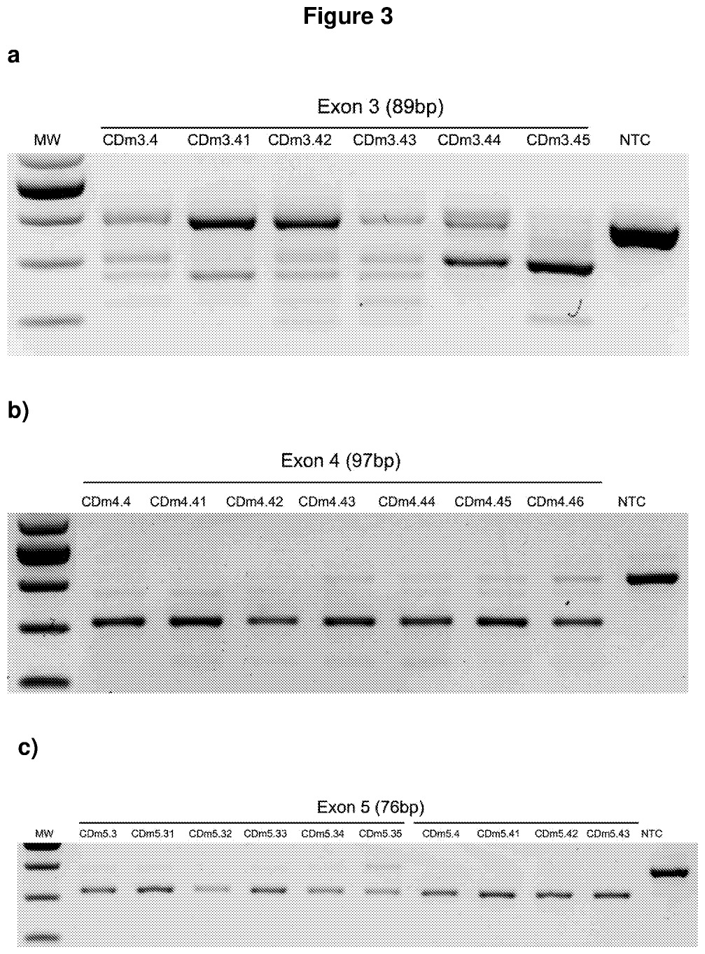

To improve the extent of exon 3 skipping, five (5) overlapping ASOs (SEQ IDs 14-18) were designed by micro-walking across the pre-mRNA region targeted by CDm3.4. The strategy involved shifting 5′ and 3′ of the region targeted by CDm3.4 by 3 bp increments while restricting the overall oligonucleotide length to 25 bases. In these experiments, which were repeated at least once, Hep G2 cells were transfected with antisense synthesised in MOE chemistry (250 nM). Total RNA was collected 24 hours post-transfection and RT-PCR analysis (using primer set SEQ 49/50) was used to assess exon-skipping (FIG. 3). A non-transfected culture (denoted NTC) was used as a wild-type mRNA control. ImageLab (Biorad) was used to quantify the percent skipping of exon 3. The best ASO sequence was CDm3.45 (SEQ ID 18) which induced almost complete skipping, while the next best ASO sequence was 3.44, which induced approx. 80% skipping.

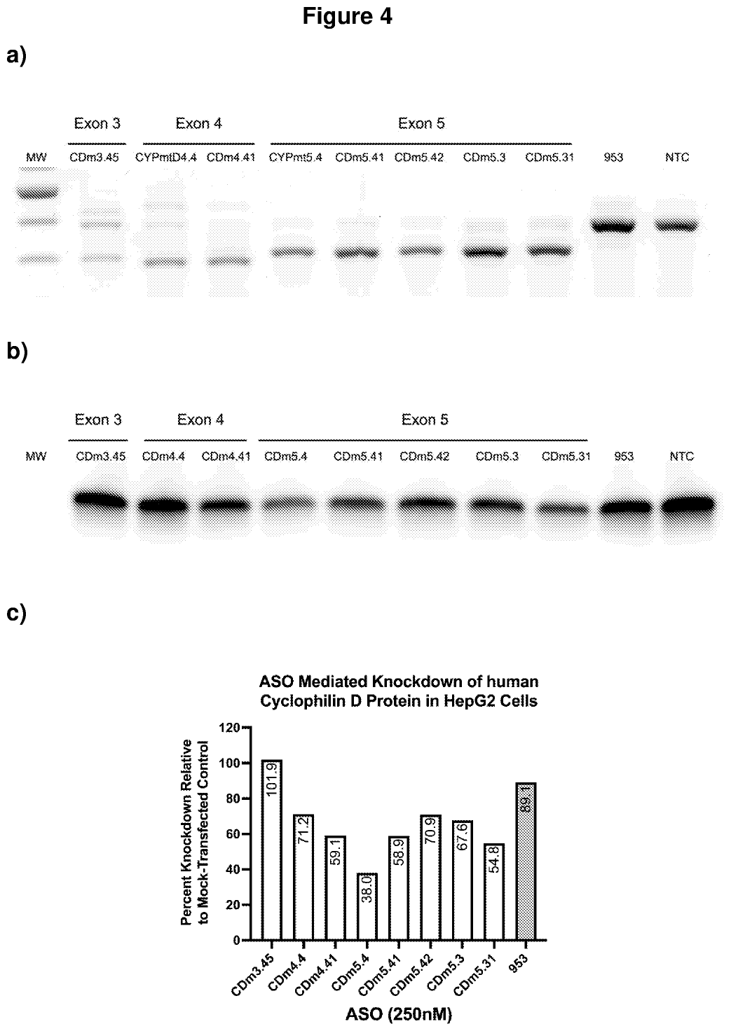

To improve the extent of exon 4 skipping, six (6) overlapping ASOs (SEQ IDs 26-31) were designed by micro-walking across the pre-mRNA region targeted by CDm4.4. The strategy involved shifting 5′ and 3′ of the region targeted by CDm4.4 in 3 bp increments while restricting the overall oligonucleotide length to 25 bases. In these experiments, which were repeated at least once, Hep G2 cells were transfected with antisense synthesised in MOE chemistry (250 nM). Total RNA was collected 24 hours post-transfection and RT-PCR analysis (using primer set SEQ 49/50) was used to assess exon-skipping (FIG. 4). A non-transfected culture (denoted NTC) was used as a wild-type mRNA control. ImageLab (Biorad) was used to quantify the percent skipping of exon 4. The best ASOs sequence were CDm4.4 (SEQ ID 22) and CDm4.41 (SEQ ID 18) which induced almost complete skipping, with no residual FL-CYPD evident. By contrast, ASOs CDm4.42-CDm4.46 induced skipping estimated at greater than 90%.

To improve the extent of exon 5 skipping, 2 sets of overlapping ASOs sequences were designed by micro-walking across the pre-mRNA region targeted by CDm5.3 (5 of: SEQ ID 37-41) and CDm5.4 (3 of: SEQ ID 42-44). The strategy involved shifting 5′ and 3′ of the region targeted by CDm5.3 and CDm5.4 in 3 bp increments while restricting the overall oligonucleotide length to 25 bases. In these experiments, which were repeated at least once, Hep G2 cells were transfected with antisense synthesised in MOE chemistry (250 nM). Total RNA was collected 24 hours post-transfection and RT-PCR analysis (using primer set SEQ 49/50) was used to assess exon-skipping (FIG. 5). A non-transfected culture (denoted NTC) was used as a wild-type mRNA control. ImageLab (Biorad) was used to quantify the percent skipping of exon 5. The ASO sequences CDm5.4, CDm5.41, CDm5.42, CDm5.43, which all induced complete skipping, with no residual FL-CYPD evident. By contrast, ASOs CDm5.3 CDm5.31, CDm5.32CD, CDm5.33, CDm5.34 induced skipping estimated to be greater than 90%, while CDm5.35 induced skipping of greater than 60%.

The best ASOs (CDm3.45, CDm4.4, CDm4.41, CDm5.3, CDm5.31 CDm5.4, CDm5.41, CDm5.42) and a scrambled control 953 (SEQ ID 45), were transfected into HepG2 cultures at 250 nM using Lipofectamine RNAiMax. Total RNA was collected 24 hours post-transfection and RT-PCR analysis (using primer set SEQ 49/50) was conducted to determine the extent of exon-skipping (FIG. 4a). Quantitation of exon skipping by each ASO was as follows: CDm3.45 induced 45.3% skipping of exon3; CDm4.4 induced 77.3% skipping of exon 4; CDm4.41 induced 92.1% skipping of exon 4; CDm5.4 induced 98.2% skipping of exon 5; CDm5.41 induced 95.2% skipping of exon 5; CDm5.42 induced 93.6% skipping of exon 5; CDm5.3 induced 94.5% skipping of exon 5; and CDm5.31 induced 93.0% skipping of exon 5. A duplicate transfection plate (in which the transfection mixture was replaced with DMEM with 1% FCS/antibiotics at 24 hours) was incubated a further 48 hours and cell lysates were harvested for Western Blot analysis. Western Blot analysis confirmed that CYPD protein was reduced at 72 hours post-transfection in all ASO treated cultures except for CDm3.45 (FIGS. 4b and 4c). Cyclophilin D protein band densities were normalised against a NTC (mock transfected) control culture (FIG. 4c). Treatment with ASOs CDm5.4 and CDm5.31 reduced CYPD expression to 38% and 54.8% respectively. Treatment with the other ASOs decreased CYPD expression, as shown in parenthesis: CDm4.4(71.2%); CDm4.41 (59.1%); CDm5.41 (58.9%); CDm5.42 (70.9%) and; CDm5.3 (67.6%). The scrambled ASO (953), which served as a treatment control, induced a 10% reduction in CYPD relative to the NTC, but the cause of this reduction in CYPD protein was not investigated, but was likely a non-specific effect on cellular protein output.

1. An isolated or purified antisense oligomer which has a modified backbone structure for modifying pre-mRNA splicing in the PPIF gene transcript or part thereof, wherein the antisense oligomer induces non-productive splicing or functional impairment in the PPIF gene transcript or part thereof, and wherein the antisense oligomer has a modified backbone structure and at least 95% sequence identity to any one of SEQ ID NOs: 1-44, and combinations or cocktails thereof.

2.-3. (canceled)

4. The antisense oligomer of claim 1, wherein the antisense oligomer contains one or more nucleotide positions subject to an alternative chemistry or modification chosen from the list comprising: (i) a modified backbone structure; (ii) modified sugar moieties; (iii) resistance to RNase H; (iv) oligomeric mimetic chemistry.

5. The antisense oligomer of claim 1 wherein the antisense oligomer is further modified by: (i) chemical conjugation to a moiety; and/or (ii) tagging with a cell penetrating peptide.

6. The antisense oligomer of claim 1, wherein when any uracil (U) is present in the nucleotide sequence, the uracil (U) is replaced by a thymine (T).

7. The antisense oligomer of claim 1, that operates to induce skipping of one or more of the exons of the PPIF gene transcript or part thereof.

8. A method for modulating splicing in a PPIF gene transcript, the method comprising
providing one or more of the antisense oligomers according to claim 1, and allowing the oligomer(s) to bind to a target nucleic acid site.

9. A pharmaceutical or therapeutic composition to treat or ameliorate the effects of a disease related to PPIF expression in a patient, the composition comprising:
a) one or more antisense oligomers according to claim 1, and
b) one or more pharmaceutically acceptable carriers and/or diluents.

10. A method to treat or ameliorate the effects of a disease associated with PPIF expression, the method comprising:
administering to the patient an effective amount of one or more antisense oligomers or pharmaceutical composition comprising one or more antisense oligomers according to claim 1.

11. (canceled)

12. A kit to treat, prevent or ameliorate the effects of a disease associated with PPIF expression in a patient, which kit comprises at least an antisense oligomer according to claim 1, packaged in a suitable container, together with instructions for its use.

13. The composition of claim 9, wherein the PPIF expression associated disease or pathology is ischaemia reperfusion related injury, oxidative related injury, inflammatory related injury and trauma related injury, neurodegeneration, diabetes, metabolic disease, skeletal muscle disease, or inflammatory disease.

14.-15. (canceled)

16. The antisense oligomer of claim 1, that operates to induce skipping of one or more of the exons of the PPIF gene transcript or part thereof.

17. The antisense oligomer of claim 1, that operates to induce skipping of exon 3, 4 or 5 of the PPIF gene transcript or part thereof.

18. The antisense oligomer of claim 1, wherein the antisense oligomer has the sequence of:
a) SEQ ID NO: 7 to 18;
b) SEQ ID NO: 19 to 31;
c) SEQ ID NO: 32 to 44; or
d) SEQ ID NO: 18, 26, 27, 35 or 37.

19. The composition of claim 9, wherein the PPIF expression associated disease or pathology is ischaemia reperfusion related injury, oxidative related injury, inflammatory related injury and trauma related injury, fibrotic disease, neurodegeneration, diabetes, metabolic disease, skeletal muscle disease, or inflammatory disease.

20. The method of claim 10, wherein the PPIF expression associated disease or pathology is ischaemia reperfusion related injury, oxidative related injury, inflammatory related injury and trauma related injury, fibrotic disease, neurodegeneration, diabetes, metabolic disease, skeletal muscle disease, or inflammatory disease.
